(19) United States
(12) Patent Application Publication
(10) Pub. No.: US 2005/0124685 A1
(43) Pub. Date: Jun. 9, 2005

(54) Polyether brevetoxin derivatives as a treatment for neurotoxic shellfish poisoning and ciguatera fish poisoning

(75) Inventors: Daniel G. Baden, Wilmington, NC (US); Kathleen S. Rein, Cooper City, FL (US); Lynne Fieber, Key Biscayne, FL (US); Sherry P. Parker, Sunrise, FL (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: University of North Carolina at Wilmington

(21) Appl. No.: 10/945,241

(22) Filed: Sep. 20, 2004

Related U.S. Application Data
(60) Provisional application No. 60/504,670, filed on Sep. 19, 2003.

(51) Int. Cl. A61K 31/335

(52) U.S. Cl. 514/450

(57) ABSTRACT

Disclosed are compounds that are derivatives of brevetoxin, or PbTx, pharmaceutical formulations comprising the compounds, and methods of administering the compounds to a subject for the treatment of neurotoxic shellfish poisoning or ciguatera fish poisoning, wherein the compounds have general Formulas (I) and (III): 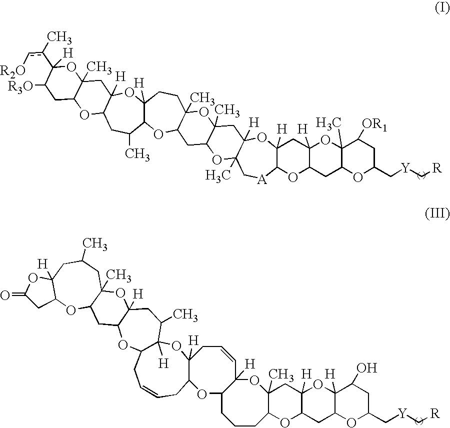
wherein R, R₁, R₂, R₃, A, n, and Y are as defined herein for each compound.

### CROSS-REFERENCE TO RELATED APPLICATIONS

This application claims priority to U.S. Provisional Application 60/504,670, filed Sep. 19, 2003.

### BACKGROUND OF THE INVENTION

1. Field of the Invention

The invention relates to brevetoxin derivatives, pharmaceutical formulations comprising the brevetoxin derivatives, and methods of treating diseases, conditions, poisoning, or illnesses derived from seafood using the compounds and pharmaceutical formulations.

2. Description of the Related Art

Florida red tides are known to have adverse effects on both marine life and humans. These tides have been linked to large fish kills, marine mammal mortality, and even human illnesses. Human illnesses caused by red tides include respiratory irritation through contact or inhalation and neurotoxic shellfish poisoning (NSP) from consumption of exposed or contaminated seafood (Purkerson-Parker, et al. 2000; Baden, D. G., et al., Toxicon, 1982; 20(5):929-932; Baden, D. G., et al., Int. Rev. Cytol., 1983; 82:99-150). Symptoms of NSP include nausea, vomiting, diarrhea, and bronchoconstriction (Purkerson-Parker, et al., Chemistry and Biology, 2000; 7(6):385-393). The causative agent in the red tide organisms has been isolated and identified as brevetoxin.

The class of compounds known as the brevetoxins were initially discovered when they were purified as toxins from cultures of the Florida red tide organism Karenia brevis also known as Gymnodinium breve and Ptychodiscus brevis (Baden, D. G., et al., Toxicon, 1982; 20(5):929-932). The brevetoxins, also known as “PbTx” toxins (Ptychodiscus brevis toxin), have since been characterized and found to be polycyclic-polyethers that initially were shown to have binding activity to a unique site associated with rat brain synaptosomes (Poli, M. A., et al., Molec. Pharm., 1986; 30:129-135). Brevetoxins are classified as neurotoxins that are believed to bind to voltage gated sodium channels. The effects of brevetoxins are mediated by interaction with receptor site 5 on the sodium channels. The general brevetoxin A and brevetoxin B backbone structure are as follows, with PbTx molecules (1-10) described.
Brevetoxin B Backbone 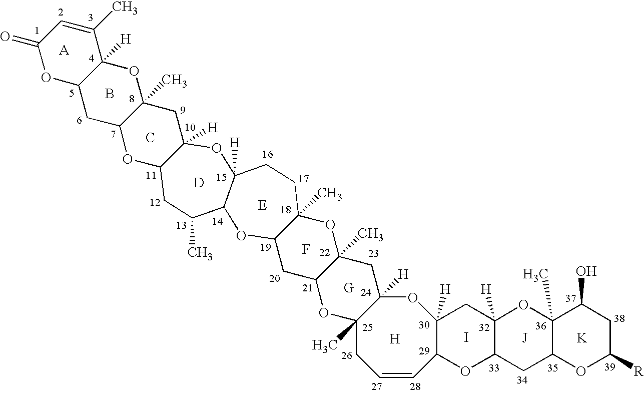
PbTx-2: R is CH₂C(═CH₂)CHO
PbTx-3: R is CH₂C(═CH₂)CH₂OH;
PbTx-5: R is CH₂C(═CH₂)CHO, and OAc (instead of OH) at C37;
PbTx-6: R is CH₂C(═CH₂)CHO, and an epoxide at C27, C28 (instead of double bond);
PbTx-8: R is CH₂COCH₂Cl
PbTx-9 R is CH₂CH(CH₃)CH₂OH.
   Brevetoxin A Backbone 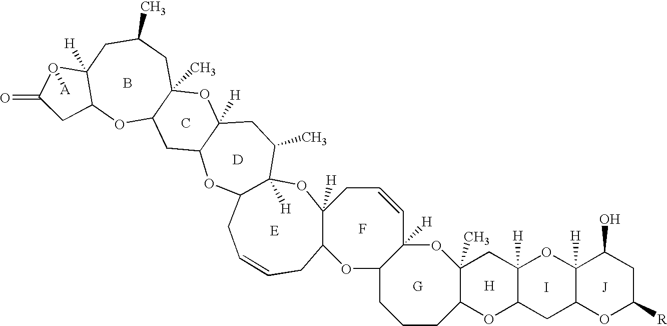
PbTx-1: R is CH₂C(═CH₂)CHO;
PbTx-7: R is CH₂C(═CH₂)CH₂OH;
PbTx-10 R is CH₂CH(CH₃)CH₂OH.

Generally, the activity of brevetoxins is thought to derive from the general backbone structure. Ring A and intact rings H, I, J, and K have been reported to be essential for the toxic activity of these compounds. There have been no reports that link toxic activity of brevetoxins to the various side chains appended to the backbone structure.

β-Naphthoyl-PbTx-3 is a brevetoxin derivative that reduces sodium channel openings and effectively antagonizes the actions of the native toxin in channel activation (Purkerson-Parker, et al., 2000). β-Naphthoyl-PbTx-3 displaces the native toxin from its binding site, does not elicit opening of sodium channels in steady state and findings indicate that it blocks brevetoxin-induced opening of sodium channels.

While the antagonist blocks the action of the toxin, its inhibition of sodium channels can be useful in the treatment of these food-borne diseases. The several brevetoxin backbone derivatives that are available, when coupled through the K-ring side chain of PbTx-2, provide for ten or more unique conjugates with differential solubility, efficacy, and potential therapeutic longevity.

Ciguatera fish poisoning (CFP) is a form of human poisoning caused by the consumption of subtropical and tropical marine fish that have accumulated naturally occurring toxins through their diet. The toxins are known to originate from several dinoflagellate (algae) species that are common to ciguatera endemic regions in the lower latitudes. Marine fish most commonly implicated in ciguatera fish poisoning include groupers, barracudas, snappers, jacks, mackerel, and triggerfish. Many other species of warm-water fish can harbor ciguatera toxins. The occurrence of toxic fish is sporadic, and not all fish of a given species or from a given locality will be toxic.

Initial signs of poisoning occur within six hours after consumption of toxic fish and typically include a combination of gastrointestinal (e.g., nausea, vomiting, and diarrhea), neurological (e.g., intensified paresthesia, arthralgia, myalgia, headache, temperature sensory reversal and acute sensitivity to temperature extremes, vertigo, and muscular weakness), and cardiovascular disorders (e.g., arrhythmia, bradycardia or tachycardia, and reduced blood pressure). Symptoms defined within these general categories vary with the geographic origin of toxic fish. Diagnosis of CFP remains unsatisfactory and is typically based on patient symptoms and recent dietary history.

Ciguatera poisoning is usually self-limiting, and signs of poisoning often subside within several days from onset. However, in severe cases the neurological symptoms are known to persist from weeks to months. In a few isolated cases neurological symptoms have persisted for several years, and in other cases recovered patients have experienced recurrence of neurological symptoms months to years after recovery. Such relapses are most often associated with changes in dietary habits or with consumption of alcohol. There is a low incidence of death resulting from respiratory and cardiovascular failure.

Current treatments for ciguatera fish poisoning are far from satisfactory. Typically intravenous administration of mannitol is used but is normally only effective if it is used in the first 48-72 hours of exposure. The treatment of chronic CFP is usually symptomatic.

CFP and NSP are thought to be induced via binding at a common receptor site on voltage gated sodium channels known as site 5. Binding by brevetoxins or ciguatoxin at site 5 results in massive influx in sodium ions at normal resting potential.

Thus, there is a need for active agents that can act as antagonists for binding of brevetoxins or ciguatoxin and/or can modulate ion and water transport across the apical membranes of epithelial cells, which are therefore useful in alleviating the neurological and gastrointestinal effects in persons affected by NSP and CFP.

The invention provides compounds, or pharmaceutically acceptable salts, solvates, hydrates, esters, amides, complexes, or combinations thereof, of Formula (I): 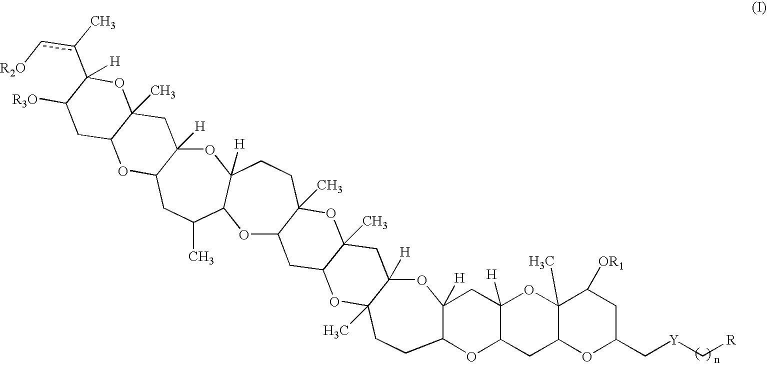
wherein
A is, 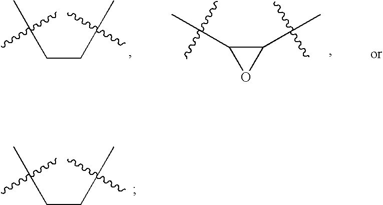
R is C₁-C₆ alkyl, C₂-C₆ alkenyl, C₁-C₆ alkyl ester, C₂-C₆ alkenyl ester, amino, amido, aryl ester, cycloalkyl ester, cycloalkenyl ester, purinyl, pyrimidinyl, heterocycle, or heteroaryl;
R₁ is H or —(CO)CH₃; and
R₂ and R₃ at each occurrence are independently —CH₂(CO)CH₃, 15-CH₂(CO)CH₂CH₃, —CH₂ (CO)CH(CH₃)₂, —CH₂ (CO)CH₂CH₂CH₃, —CH₂ (CO)CH(CH₃) CH₂CH₃, or —CH₂ (CO)CH₂CH(CH₃)₂,
   or OR₂ and OR₃ can be taken together to form a six membered ring of the formula (Ia) 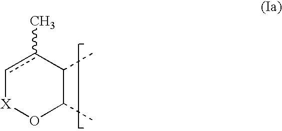
   wherein X is C═O or CH(CH₃);
   wherein the bracketed-dashed bonds indicate attachment to backbone;
Y is C═CH₂, C═O, CHCH₃, or CH₂; and
n is 1 or 0; and
   with the proviso that when OR₂ and OR₃ are taken together to form a ring of the formula (Ia), wherein X is C═O and the double bond is present; when A is 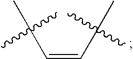
   and when n is 1, R is not:

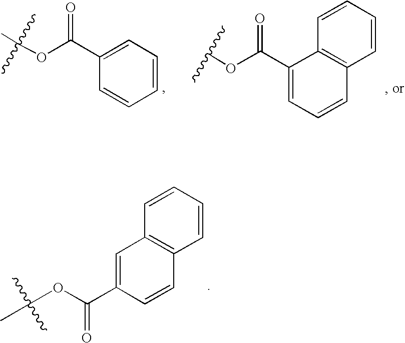

The invention also provides compounds, or pharmaceutically acceptable salts, solvates, esters, amides, hydrates, complexes, or combinations thereof, of the Formula (II): 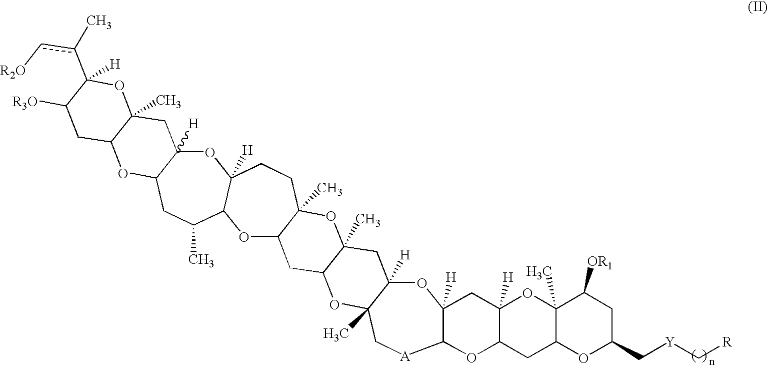
wherein
A is 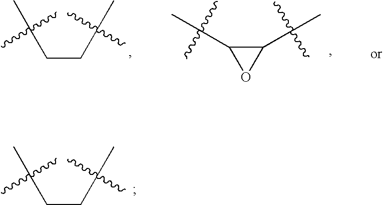
R is C₁-C₆ alkyl, C₂-C₆ alkenyl, C₁-C₆ alkyl esters, C₂-C₆ alkenyl ester, amino, amides, aryl ester, cycloalkyl ester, cycloalkenyl ester, purinyl, pyrimidinyl, heterocyclyl, or heteroaryl;
R₁ is H or —COCH₃; and
R₂ and R₃ at each occurrence are independently —CH₂COCH₃, —CH₂COCH₂CH₃, —CH₂COCH(CH₃)₂, —CH₂COCH₂CH₂CH₃, —CH₂COCH(CH₃) CH₂CH₃, or —CH₂COCH₂CH(CH₃)₂,
   or OR₂ and OR₃ can be taken together to form a six membered ring of the formula (Ia) 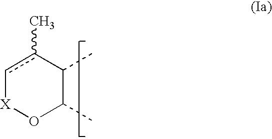
   wherein X is C═O or CH(CH₃);
   wherein the bracketed-dashed bonds indicate attachment to backbone;
Y is C═CH₂, C═O, or CH₂; and
n is 1 or 0; and
   with the proviso that when OR₂ and OR₃ are taken together to form a ring of the formula (Ia), wherein X is C═O and the double bond is present; when A is 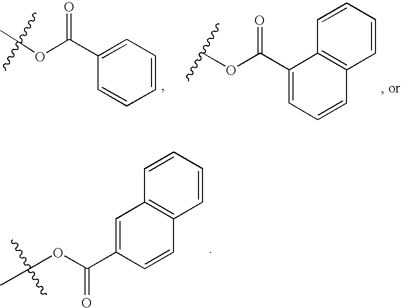
   and when n is 1, R is not:

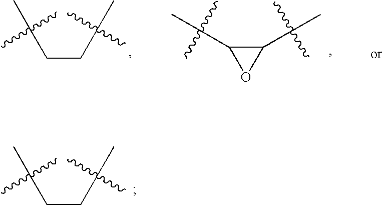

Further, the invention provides compounds, or pharmaceutically acceptable salts, amides, esters, solvates, hydrates, complexes, or combinations thereof, of the Formula (III): 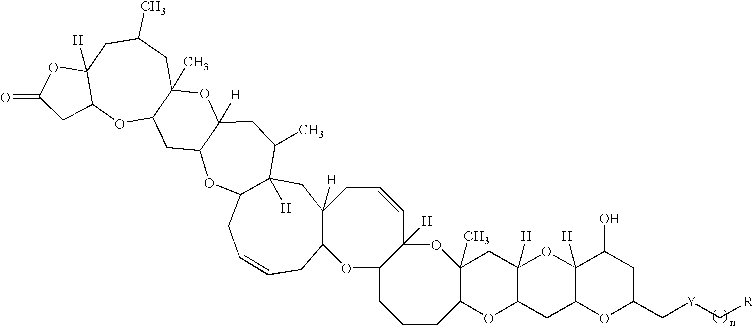
wherein
R is H, OH, halogen, C₁-C₆ lower alkyl, C₁-C₆ alkyl ester, C₂-C₆ alkenyl ester, amino, amides, aldehydo, aryl ester, cycloalkyl ester, cycloalkenyl ester, purines, pyrimidinyl, heterocyclyl, or heteroaryl;
n is 1 or 0; and
Y is C═O, C═CH₂, CHCH₃ or CH₂.

Yet further, the instant invention provides compounds, or pharmaceutically acceptable salts, amides, esters, solvates, hydrates, complexes, or combinations thereof, of the Formula (IV): 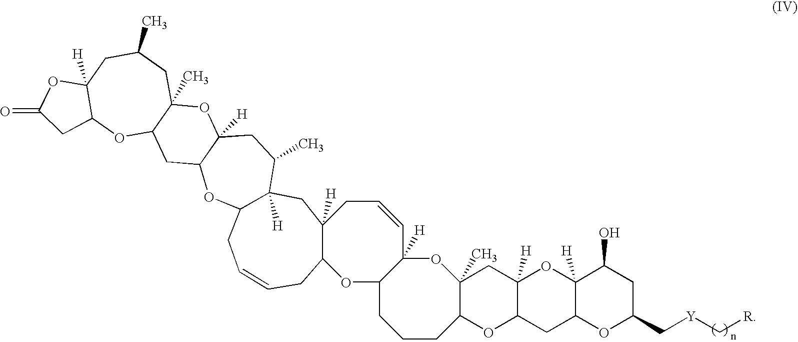
wherein
R is H, OH, halogen, C₁-C₆ lower alkyl, C₁-C₆ alkyl esters, C₂-C₆ alkenyl ester, amino, amido, aldehydo, aryl ester, cycloalkyl ester, cycloalkenyl ester, purinyl, pyrimidinyl, heterocyclyl, or heteroaryl;
n is 1 or 0; and
Y is C═CH₂, C═O, CH(CH₃), or CH₂.

The invention also provides pharmaceutical formulations comprising a compound, or a pharmaceutically acceptable salt, solvate, hydrate, complex, or combinations thereof, of Formulas (I), (II), (III), and (IV) in combination with a pharmaceutically acceptable carrier, excipient, solvent, adjuvant or diluent.

The invention further provides methods of treating neurotoxic shellfish poisoning (NSP) and/or ciguatera fish poisoning (CFP) in a subject comprising administering to a subject a compound, of Formula (I), (II), (III), or (IV), or pharmaceutically acceptable salt, solvate, hydrate, complex, or combination thereof.

### DETAILED DESCRIPTION OF THE INVENTION

### BRIEF DESCRIPTION OF THE FIGURES

FIG. 1 is a graph that illustrates the maximum binding activity in competitive displacement assays of brevetoxin PbTx-3 and its benzoyl, a-naphthoyl, and β-naphthoyl derivatives in isolated rat brain sodium channels, as observed in synaptosomal binding experiments. The Ki values for each are 3.00 nM (PbTx-3), 4.68 nM (α-naphthoyl), 1.06 nM (β-naphthoyl), and 0.20 nM (benzoyl).

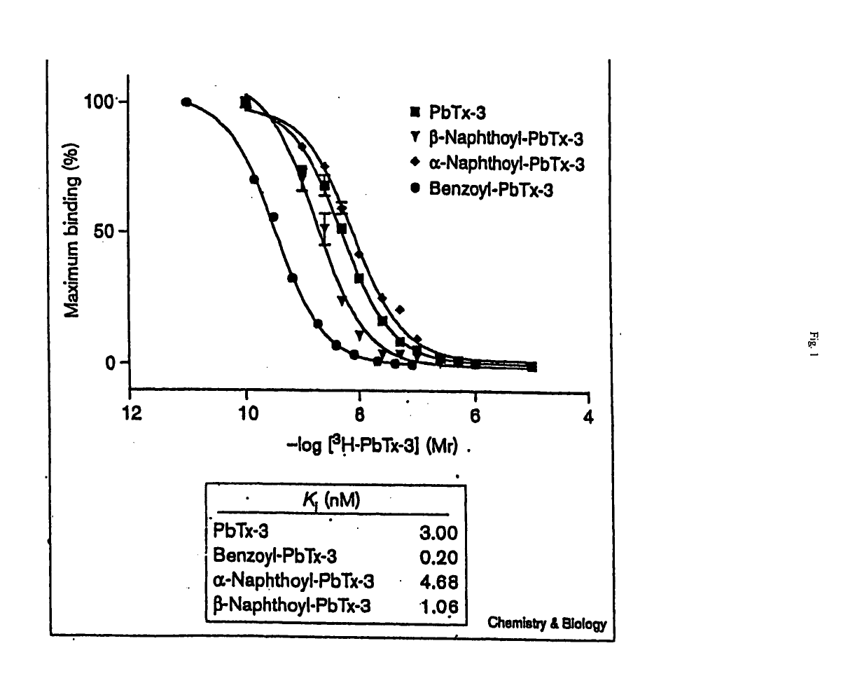

FIG. 2A-D illustrates single sodium channel currents in patch clamp sodium channels during successive command depolarizations to −10 mV from a command potential of −100 mV in the same membrane patch before (2A) and after (2B) exposure to β-naphthoyl-PbTx-3 (100 nM, n=6). FIGS. 2C and 2D: the ensemble averages indicated a reduction in peak sodium current. The average area under the β-naphthoyl-PbTx-3 curve was 71.8±18.3% less than that of PbTx-3.

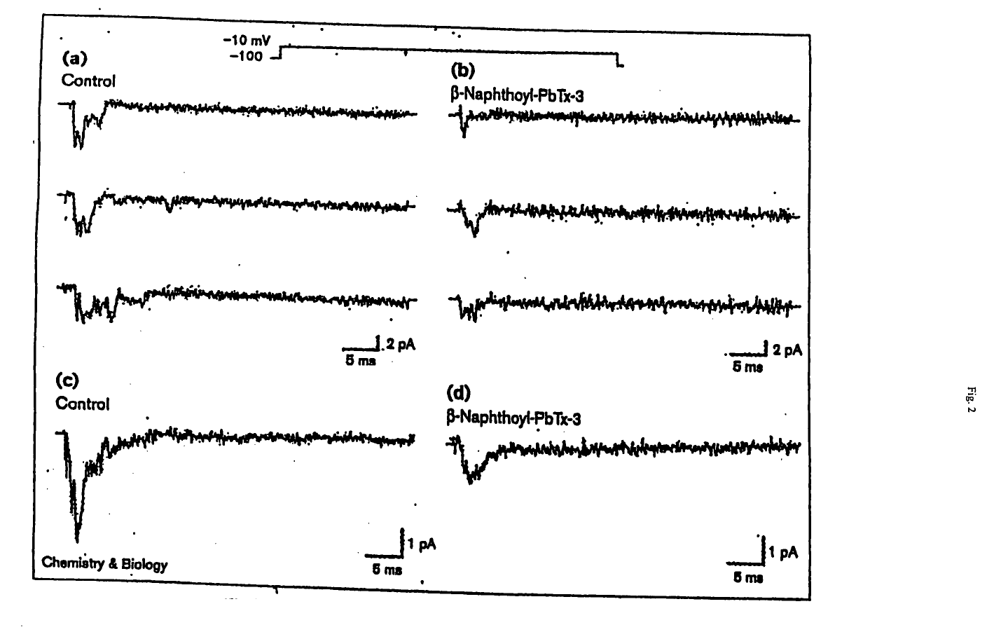

FIG. 3A-E illustrates the steady state currents in four different membrane patches at a steady state command voltage of −50 mV. 3A are control traces preceeding the test with PbTx-3, and are representative of control recordings observed in all four patches during 7.2 s of continuous observation. 3B are traces for membrane patches exposed to PbTx-3 (100 nM, n=50). 3C are traces for membrane patches exposed to benzoyl-PbTx-3 (100 nM, n=6). 3D are traces for membrane patches exposed to α-naphthoyl-PbTx-3 (100 nM, n=6). 3E are traces for membrane patches exposed to β-naphthoyl-PbTx-3 (100 nM, n=6). The experimental recordings are representative of 7.2 s of continuous observation in PbTx-3 or the particular derivative.

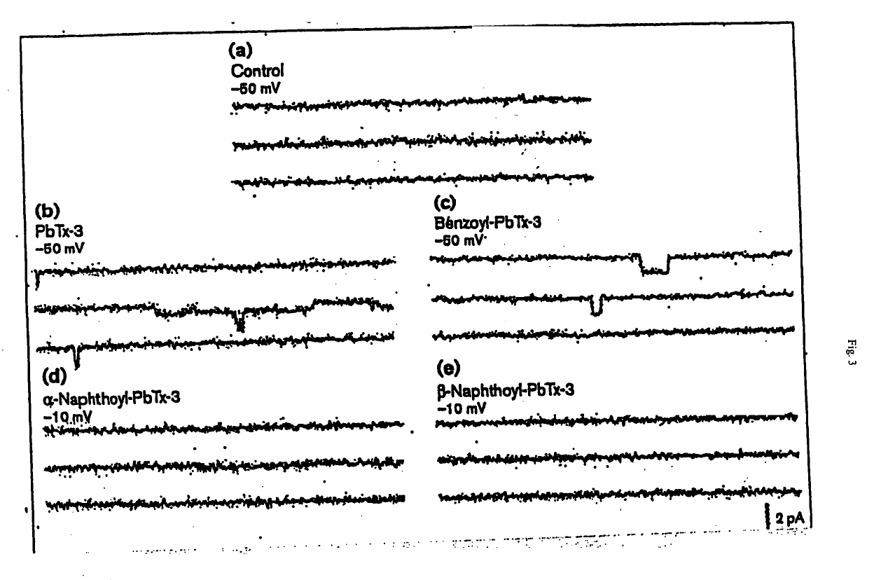

Definitions

Unless defined otherwise, all scientific and technical terms used herein have the same meaning as commonly understood by one of skill in the art to which this invention belongs.

All patents and publications referred to herein are hereby incorporated by reference for all purposes.

A “therapeutically effective” amount is defined as an amount effective to reduce or lessen at least one symptom of the disease being treated or to reduce or delay onset of one or more clinical markers or symptoms of the disease.

As used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

By “alkyl” and “C₁-C₆ alkyl” is meant straight or branched chain alkyl groups having 1-6 carbon atoms, such as, methyl, ethyl, propyl, isopropyl, n-butyl, sec-butyl, tert-butyl, pentyl, 2-pentyl, isopentyl, neopentyl, hexyl, 2-hexyl, 3-hexyl, and 3-methylpentyl. It is understood that in cases where an alkyl chain of a substituent (e.g. of an alkyl, alkoxy or alkenyl group) is shorter or longer than 6 carbons, it will be so indicated in the second “C” as, for example, “C₁-C₁₀” indicates a maximum of 10 carbons. The alkyl groups herein are optionally substituted in one or more substitutable positions with various groups. Preferred alkyl groups are optionally substituted with C₁-C₆ alkoxy, halogen, hydroxy, cyano, nitro, amino, mono (C₁-C₆) alkylamino, di (C₁-C₆)alkylamino, C₁-C₆ haloalkyl, or C₁-C₆ haloalkoxy.

By the term “halogen” is meant fluorine, bromine, chlorine, and iodine.

“Alkenyl” and “C₂-C₆ alkenyl” means straight and branched hydrocarbon groups having from 2 to 6 carbon atoms and from one to three double bonds and includes, for example, ethenyl, propenyl, 1-but-3-enyl, 1-pent-3-enyl, 1-hex-5-enyl and the like. The alkenyl groups herein are optionally substituted in one or more substitutable positions with various groups.

As used herein, the term “cycloalkyl” refers to saturated carbocyclic groups having three to twelve carbon atoms. The cycloalkyl can be monocyclic, or a polycyclic fused system. Examples of such groups include cyclopropyl, cyclobutyl, cyclopentyl and cyclohexyl. The cycloalkyl groups herein are unsubstituted or, as specified, substituted in one or more substitutable positions with various groups. Preferred cycloalkyl groups are optionally substituted with C₁-C₆ alkyl, C₁-C₆ alkoxy, halogen, hydroxy, cyano, nitro, amino, mono(C₁-C₆) alkylamino, di (C₁-C₆) alkylamino, C₂-C₆alkenyl, C₂-C₆alkynyl, C₁-C₆ haloalkyl, C₁-C₆ haloalkoxy, amino (C₁-C₆) alkyl, mono (C₁-C₆) alkylamino (C₁-C₆) alkyl or di (C₁-C₆) alkylamino (C₁-C₆) alkyl.

By “aryl” is meant an aromatic carbocyclic group having a single ring (e.g., phenyl), multiple rings (e.g., biphenyl), or multiple condensed rings in which at least one is aromatic, (e.g., 1,2,3,4-tetrahydronaphthyl, naphthyl), which is optionally mono-, di-, or trisubstituted. Preferred aryl groups of the invention are phenyl, 1-naphthyl, 2-naphthyl, indanyl, indenyl, dihydronaphthyl, tetralinyl or 6,7,8,9-tetrahydro-5H-benzo[α]cycloheptenyl. The aryl groups herein are unsubstituted or, as specified, substituted in one or more substitutable positions with various groups. Preferred aryl groups are optionally substituted with, C₁-C₆ alkyl, C₁-C₆ alkoxy, halogen, hydroxy, cyano, nitro, amino, mono(C₁-C₆) alkylamino, di (C₁-C₆) alkylamino, C₂-C₆alkenyl, C₂-C₆alkynyl, C₁-C₆ haloalkyl, C₁-C₆ haloalkoxy, amino(C₁-C₆)alkyl, mono(C₁-C₆)alkylamino(C₁-C₆)alkyl or di(C₁-C₆)alkylamino(C₁-C₆)alkyl.

As used herein, the term “arylester” encompasses aryloxycarbonyl and arylcarbonyloxy groups.

As used herein, the term “alkylester” encompasses alkyloxycarbonyl and alkylcarbonyloxy groups. As used herein, alkylcarbonyl carries the same meaning as alkanoyl.

As used herein, the term “alkylamide” encompasses alkylaminocarbonyl groups, dialkylcarbonyl groups, and alkanoylamino groups.

As used herein, the term “alkenylamide” encompasses alkenylaminocarbonyl groups, dialkenylcarbonyl groups, and alkenylcarbonylamino groups.

As used herein, the term “alkenylester” encompasses alkenyloxycarbonyl and alkenylcarbonyloxy groups.

The term alkylarylester as used herein refers to alkyloxycarbonyl and akanoyloxy groups in which the alkyl portion carries an aryl or heteroaryl group.

The term alkenylarylester as used herein refers to alkenyloxycarbonyl and alkenylcarbonyloxy groups in which the alkenyl portion carries an aryl or heteroaryl group.

The term cycloalkylester as used herein refers to (cycloalkyl)oxycarbonyl, i.e, where the ester is formed by reacting an acid and an cycloalkyl alcohol, and cycloalkylcarbonyloxy groups.

By “heteroaryl” is meant one or more aromatic ring systems of 5-, 6-, or 7-membered rings which includes fused ring systems of 9-11 atoms containing at least one and up to four heteroatoms selected from nitrogen, oxygen, or sulfur. Preferred heteroaryl groups of the invention include pyridinyl, pyrimidinyl, quinolinyl, benzothienyl, indolyl, indolinyl, pryidazinyl, pyrazinyl, isoindolyl, isoquinolyl, quinazolinyl, quinoxalinyl, phthalazinyl, imidazolyl, isoxazolyl, pyrazolyl, oxazolyl, thiazolyl, indolizinyl, indazolyl, benzothiazolyl, benzimidazolyl, benzofuranyl, furanyl, thienyl, pyrrolyl, oxadiazolyl, thiadiazolyl, triazolyl, tetrazolyl, oxazolopyridinyl, imidazopyridinyl, isothiazolyl, naphthyridinyl, cinnolinyl, carbazolyl, beta-carbolinyl, isochromanyl, chromanyl, tetrahydroisoquinolinyl, isoindolinyl, isobenzotetrahydrofuranyl, isobenzotetrahydrothienyl, isobenzothienyl, benzoxazolyl, pyridopyridinyl, benzotetrahydrofuranyl, benzotetrahydrothienyl, purinyl, benzodioxolyl, triazinyl, phenoxazinyl, phenothiazinyl, pteridinyl, benzothiazolyl, imidazopyridinyl, imidazothiazolyl, dihydrobenzisoxazinyl, benzisoxazinyl, benzoxazinyl, dihydrobenzisothiazinyl, benzopyranyl, benzothiopyranyl, coumarinyl, isocoumarinyl, chromonyl, chromanonyl, pyridinyl-N-oxide, tetrahydroquinolinyl, dihydroquinolinyl, dihydroquinolinonyl, dihydroisoquinolinonyl, dihydrocoumarinyl, dihydroisocoumarinyl, isoindolinonyl, benzodioxanyl, benzoxazolinonyl, pyrrolyl N-oxide, pyrimidinyl N-oxide, pyridazinyl N-oxide, pyrazinyl N-oxide, quinolinyl N-oxide, indolyl N-oxide, indolinyl N-oxide, isoquinolyl N-oxide, quinazolinyl N-oxide, quinoxalinyl N-oxide, phthalazinyl N-oxide, imidazolyl N-oxide, isoxazolyl N-oxide, oxazolyl N-oxide, thiazolyl N-oxide, indolizinyl N-oxide, indazolyl N-oxide, benzothiazolyl N-oxide, benzimidazolyl N-oxide, pyrrolyl N-oxide, oxadiazolyl N-oxide, thiadiazolyl N-oxide, triazolyl N-oxide, tetrazolyl N-oxide, benzothiopyranyl S-oxide, benzothiopyranyl S,S-dioxide. The heteroaryl groups herein are unsubstituted or, as specified, substituted in one or more substitutable positions with various groups. Preferred heteroaryl groups are optionally substituted with C₁-C₆ alkyl, C₁-C₆ alkoxy, halogen, hydroxy, cyano, nitro, amino, mono (C₁-C₆) alkylamino, di (C₁-C₆) alkylamino, C₂-C₆alkenyl, C₂-C₆alkynyl, C₁-C₆ haloalkyl, C₁-C₆ haloalkoxy, amino(C₁-C₆)alkyl, mono(C₁-C₆)alkylamino(C₁-C₆)alkyl or di(C₁-C₆)alkylamino(C₁-C₆)alkyl. Preferred heteroaryl groups include optionally substituted purine and pyrimidine groups.

By “heterocycle”, “heterocycloalkyl” or “heterocyclyl” is meant one or more carbocyclic ring systems of 4-, 5-, 6-, or 7-membered rings which includes fused ring systems of 9-11 atoms containing at least one and up to four heteroatoms selected from nitrogen, oxygen, or sulfur. Preferred heterocycles of the invention include morpholinyl, thiomorpholinyl, thiomorpholinyl S-oxide, thiomorpholinyl S,S-dioxide, piperazinyl, homopiperazinyl, pyrrolidinyl, pyrrolinyl, tetrahydropyranyl, piperidinyl, tetrahydrofuranyl, tetrahydrothienyl, homopiperidinyl, homomorpholinyl, homothiomorpholinyl, homothiomorpholinyl S,S-dioxide, oxazolidinonyl, dihydropyrazolyl, dihydropyrrolyl, dihydropyrazinyl, dihydropyridinyl, dihydropyrimidinyl, dihydrofuryl, dihydropyranyl, tetrahydrothienyl S-oxide, tetrahydrothienyl S,S-dioxide and homothiomorpholinyl S-oxide. The heterocycle groups herein are unsubstituted or, as specified, substituted in one or more substitutable positions with various groups. For example, such heterocycle groups may be optionally substituted with, for example, C₁-C₆ alkyl, C₁-C₆ alkoxy, halogen, hydroxy, cyano, nitro, amino, mono(C₁-C₆) alkylamino, di (C₁-C₆) alkylamino, C₂-C₆alkenyl, C₂-C₆alkynyl, C₁-C₆ haloalkyl, C₁-C₆ haloalkoxy, amino(C₁-C₆)alkyl, mono(C₁-C₆) alkylamino (C₁-C₆) alkyl, di (C₁-C₆) alkylamino (C₁-C₆) alkyl or ═O.

As used herein, the terms “treatment” and “treating” encompass prophylactic administration of the compound or a pharmaceutical composition comprising the compound (“prophylaxis”) as well as remedial therapy to reduce or eliminate a disease or disorder mentioned herein. Prophylactic administration may be used for a subject that is at risk of having, e.g., shellfish poisoning, while remedial therapy would be employed in situations where the subject has contracted a disease or disorder mentioned herein, e.g., shellfish poisoning or ciguatera fish poisoning. Prophylactic administration is intended for preventing disorders or preventing recurrence of disorders and may be used to treat a subject that is at risk of having or suffering from one or more disorders mentioned herein. Thus, as used herein, the term “treatment”, or a derivative thereof, contemplates partial or complete inhibition of the stated disease state, when an active ingredient of the invention is administered prophylactically or following the onset of the disease state for which such active ingredient of the is administered. “Prophylaxis” refers to administration of the active ingredient(s) to a mammal to protect the mammal from any of the disorders set forth herein, as well as others.

As used herein, the term “subject” encompasses animals, including mammals and fish. Preferably the term refers to mammals such as a humans, cattle and horses, more preferably to humans and domestic animals such as cats, dogs, and horses, and most preferably to humans.

In one aspect, the invention relates to compounds of Formula (I): 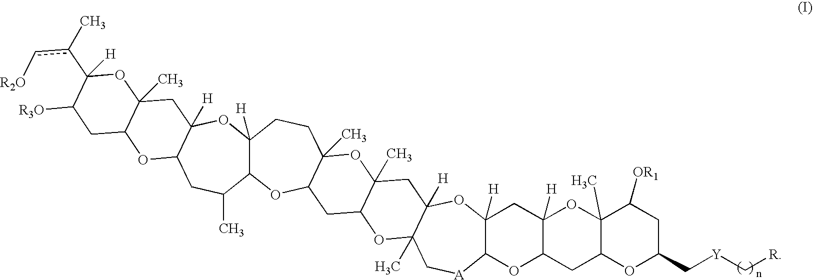
wherein
A is 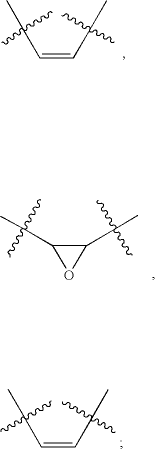
R is C₁-C₆ alkyl, C₂-C₆ alkenyl, C₁-C₆ alkyl esters, C₂-C₆ alkenyl esters, amines, amides, aryl esters, cycloalkyl esters, cycloalkenyl esters, purines, pyrimidines, heterocycle, or heteroaryl;
R₁ is H or —(CO)CH₃; and
R₂ and R₃ at each occurrence are independently —CH₂(CO)CH₃, —CH₂ (CO)CH₂CH₃, —CH₂ (CO)CH(CH₃)₂, —CH₂ (CO)CH₂CH₂CH₃, —CH₂ (CO)CH(CH₃)CH₂CH₃, or —CH₂(CO)CH₂CH(CH₃)₂,
   or OR₂ and OR₃ can be taken together to form a six membered ring of the formula (Ia) 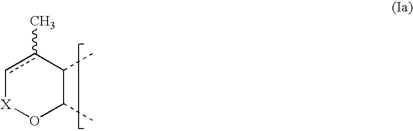
   wherein X is C═O or CH(CH₃);
wherein the bracketed-dashed bonds indicate attachment to backbone; and
Y is C═CH₂, C═O, CHCH₃, or CH₂;
n is 1 or 0; and
   with the proviso that when OR₂ and OR₃ are taken together to form a ring of the formula (Ia), wherein X is C═O and the double bond is present; when A is 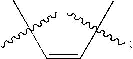
   and when n is 1, R is not: 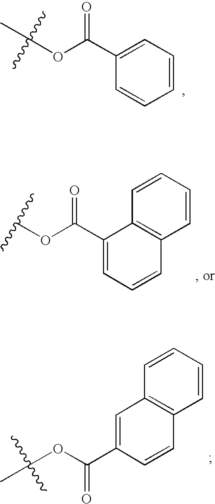
   or a pharmaceutically acceptable salt, solvate, hydrate, complex, or combinations thereof.

In a broad aspect, R is alkyl, alkyl ester, halogen, alkenyl, alkenyl ester, cycloalkyl, cycloalkyl ester, aryl, aryl ester, heteroaryl, heterocycle, heterocycloalkyl or heterocyclyl.

In another embodiment of this aspect, the compound is of Formula (I), wherein R is

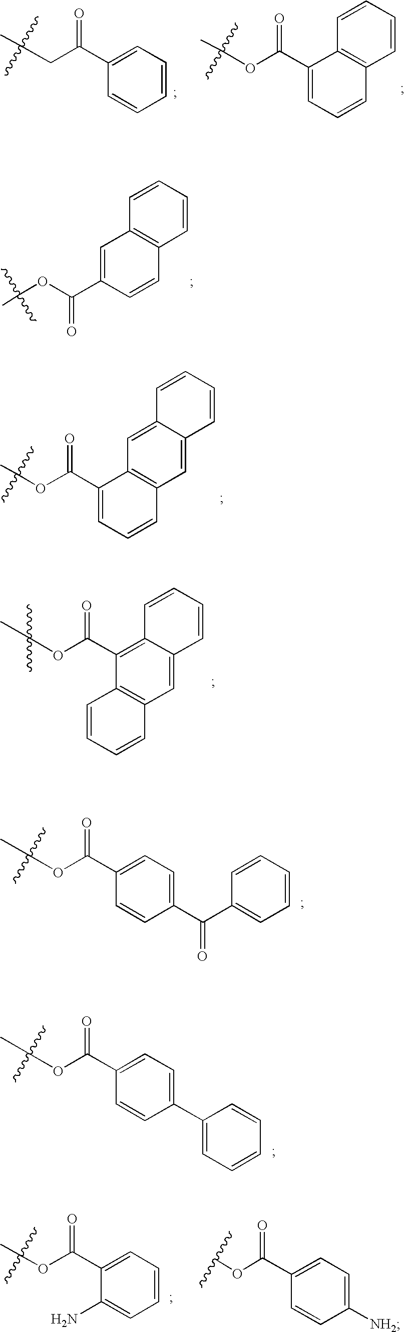

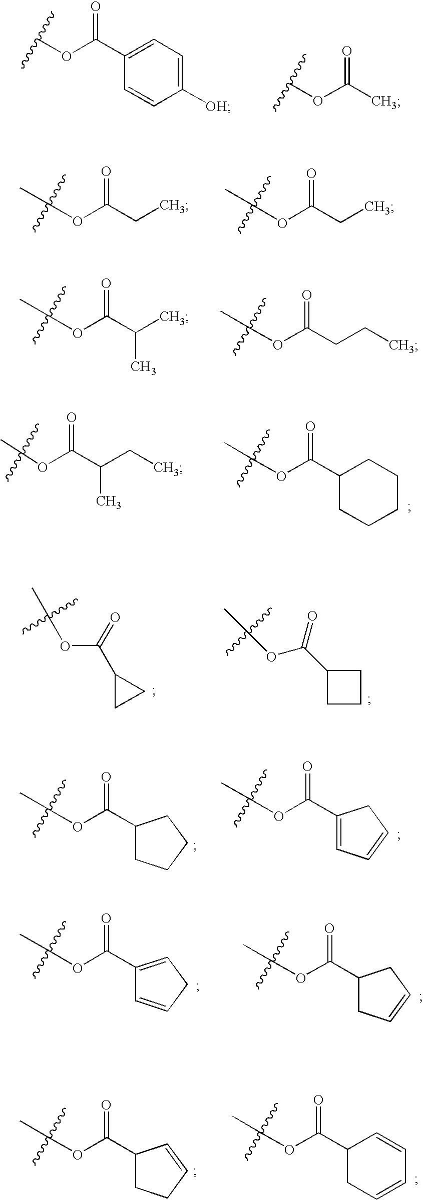

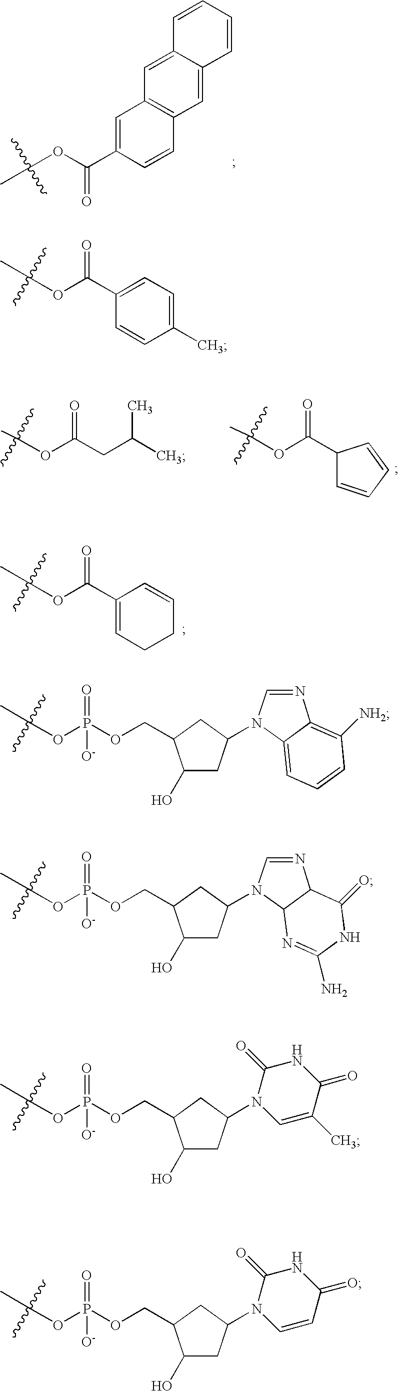

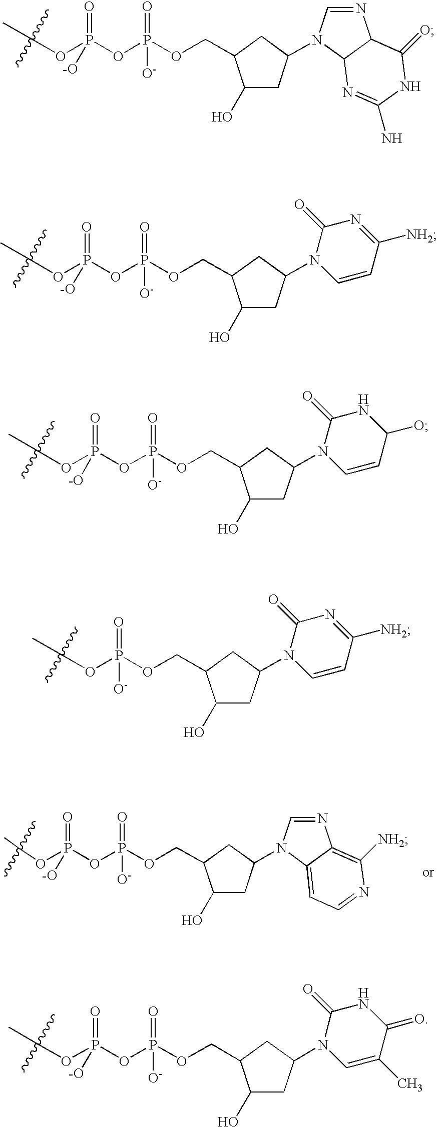

In a further embodiment of this aspect, the compound is of Formula (I), wherein R is benzoyl, α-naphthoyl, β-naphthoyl, α-anthracoyl, β-anthracoyl, or γ-anthracoyl.

In another embodiment of this aspect, OR₂ and OR₃ are taken together to form a ring of formula (Ia), wherein the ring is 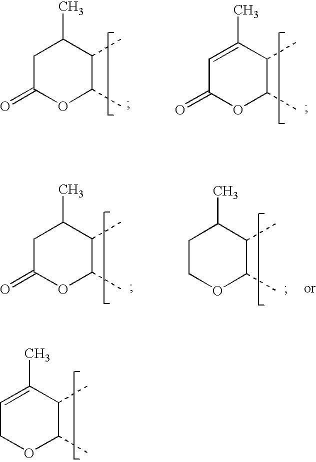
wherein the bracketed-dashed bonds indicate the point of attachment to the backbone.

In even another embodiment of this aspect, R₂ and R₃ are each independently

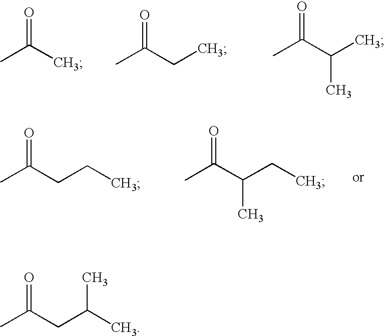

In a preferred embodiment, the invention provides compounds of Formula (II): 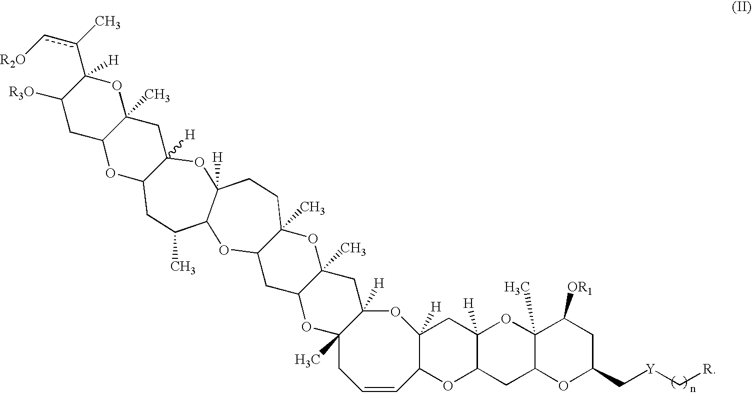
wherein
A is 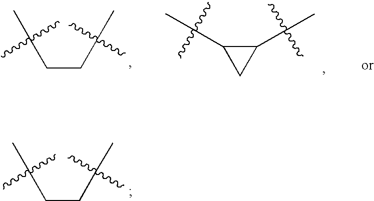
R is C₁-C₆ alkyl, C₂-C₆ alkenyl, C₁-C₆ alkyl esters, C₂-C₆ alkenyl esters, amines, amides, aryl esters, cycloalkyl esters, cycloalkenyl esters, purines, pyrimidines, heterocycle, or heteroaryl;
R₁ is H or —COCH₃; and
R₂ and R₃ at each occurrence are independently —CH₂COCH₃, —CH₂COCH₂CH₃, —CH₂COCH(CH₃) 2, —CH₂COCH₂CH₂CH₃, —CH₂COCH(CH₃) CH₂CH₃, or —CH₂COCH₂CH(CH₃)₂,
   or OR₂ and OR₃ can be taken together to form a six membered ring of the formula (Ia) 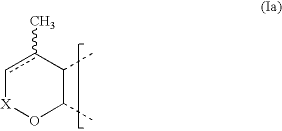
   wherein X is C═O or CH(CH₃);
wherein the bracketed-dashed bonds indicate attachment to backbone;
Y is C═CH₂, C═O, or CH₂; and
n is 1 or 0; and
   with the proviso that when OR₂ and OR₃ are taken together to form a ring of the formula (Ia), wherein X is C═O and the double bond is present; when A is 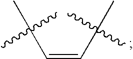
   and when n is 1, R is not: 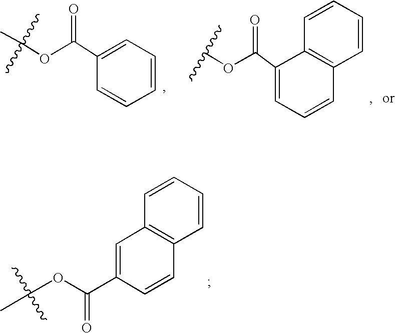
   or a pharmaceutically acceptable salt, solvate, hydrate, complex, or combination thereof.

In a further preferred embodiment, the compound of Formula (I) is:

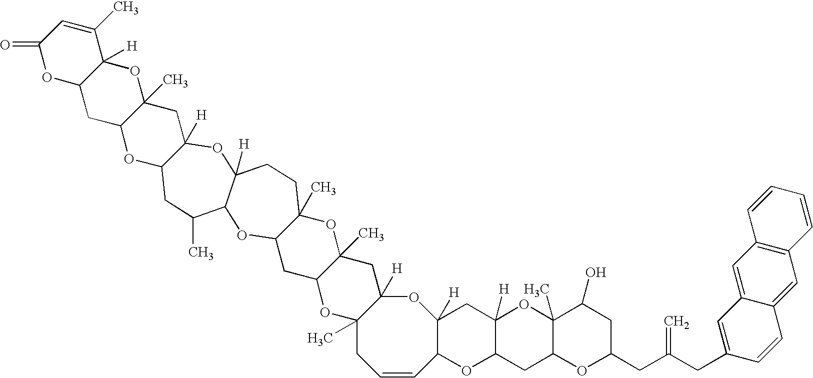

In one aspect of this preferred embodiment, the compound of Formula (I) is:

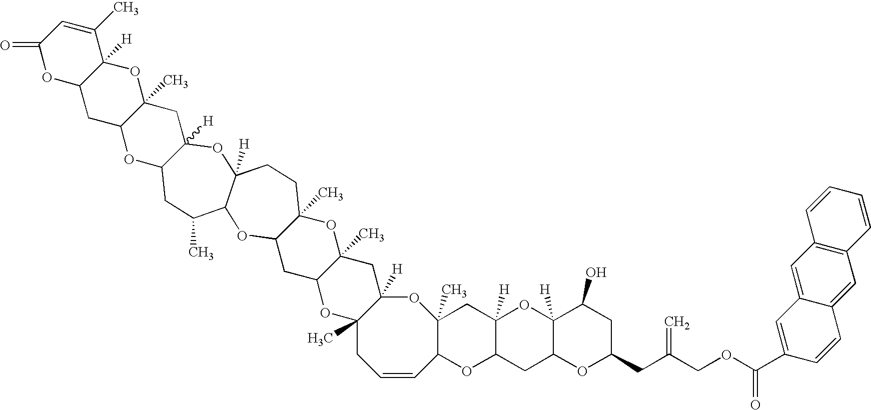

In a further preferred embodiment, the compound of Formula (I) is:

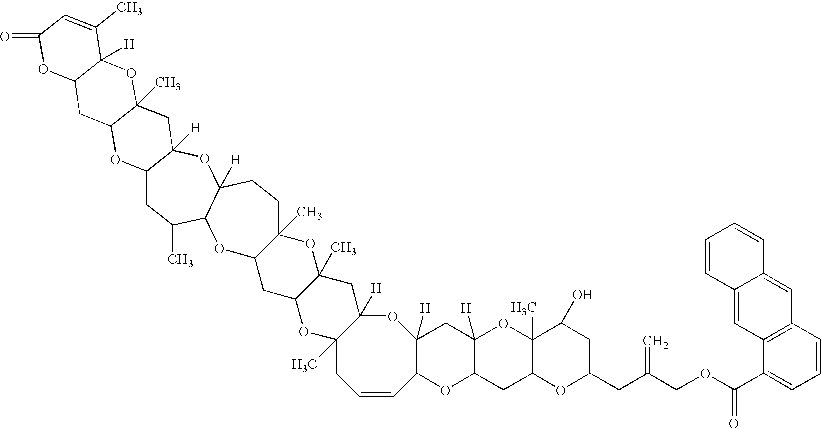

In one aspect of this preferred embodiment, the compound of Formula (I) is:

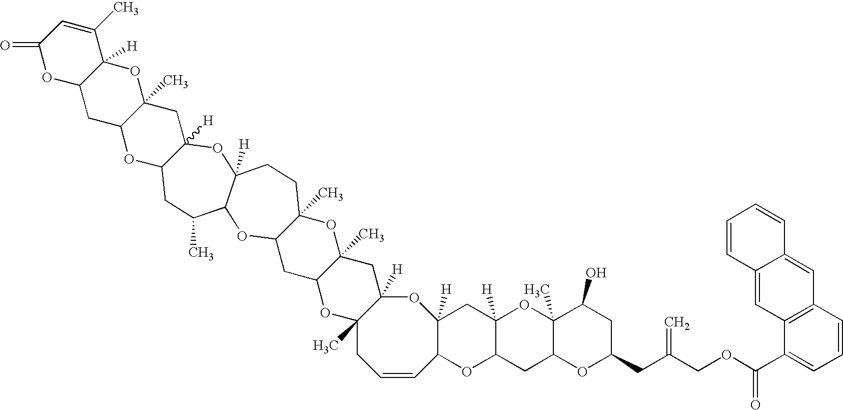

In a further preferred embodiment, the compound of Formula (I) is:

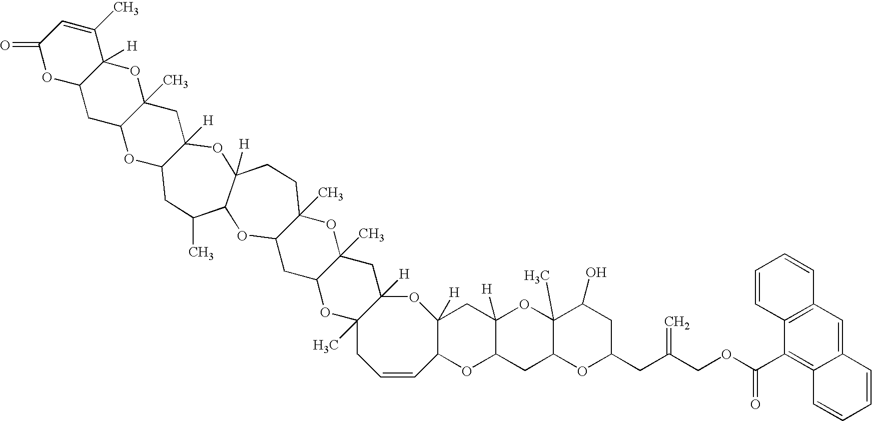

In one aspect of this preferred embodiment, the compound of Formula (I) is:

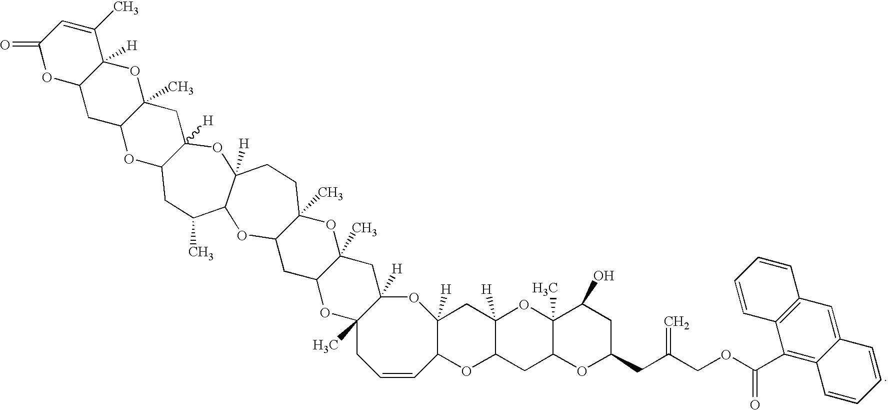

The invention also relates to compounds, or pharmaceutically acceptable salts, solvates, hydrates, complexes, or combination thereof, of Formula (III): 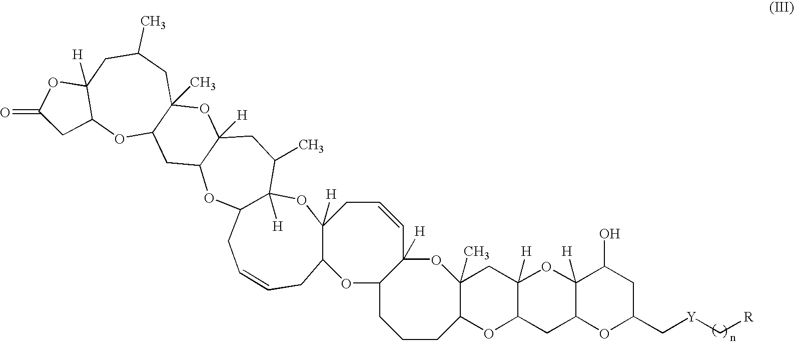
wherein
R is H, OH, halogen, C₁-C₆ lower alkyl, C₁-C₆ alkyl esters, C₂-C₆ alkenyl esters, amines, amides, aldehyde, aryl esters, cycloalkyl esters, cycloalkenyl esters, purines, pyrimidines, heterocycle, or heteroaryl;
Y is C═O, C═CH₂, CHCH₃ or CH₂; and
n is 1 or 0.

In one embodiment of this aspect, the compound of formula (III) is of formula (IV): 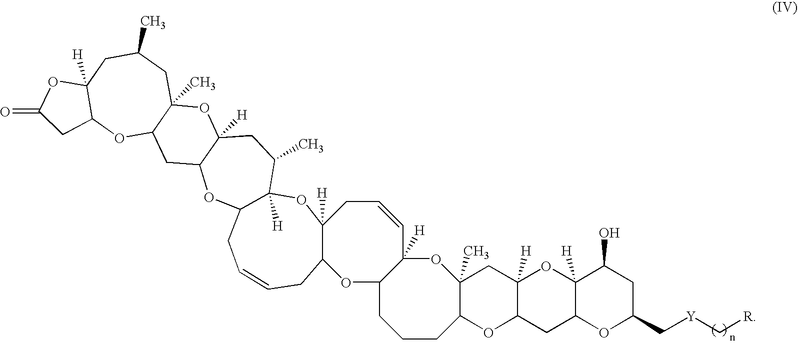
wherein
R is H, OH, halogen, C₁-C₆ lower alkyl, C₁-C₆ alkyl esters, C₂-C₆ alkenyl esters, amines, amides, aldehyde, aryl esters, cycloalkyl esters, cycloalkenyl esters, purines, pyrimidines, heterocycle, or heteroaryl;
Y is C═O, C═CH₂, CHCH₃ or CH₂; and
n is 1 or 0.

In a preferred embodiment the compound of formula (III) is:

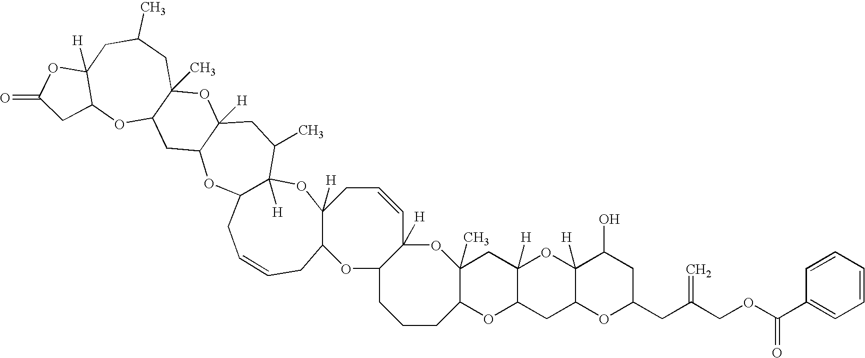

In one aspect of this preferred embodiment, the compound is:

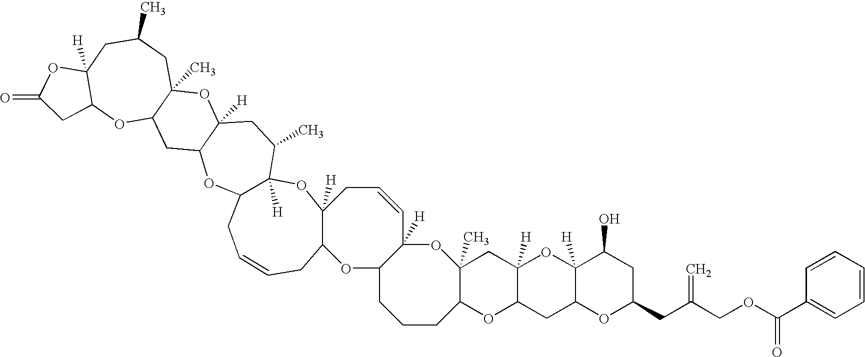

In another preferred embodiment the compound of formula (III) is:

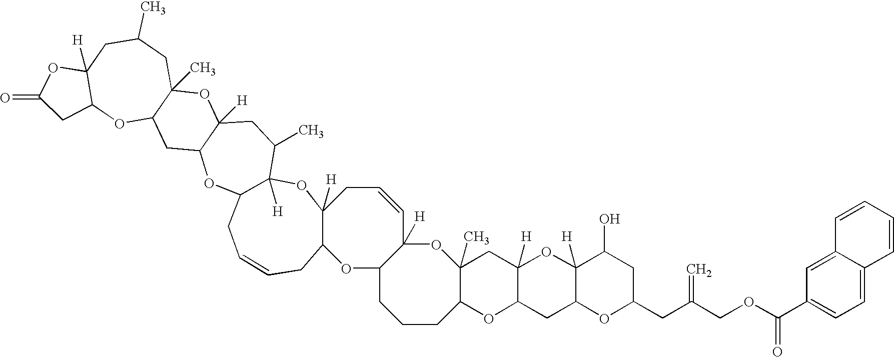

In one aspect of this preferred embodiment, the compound is:

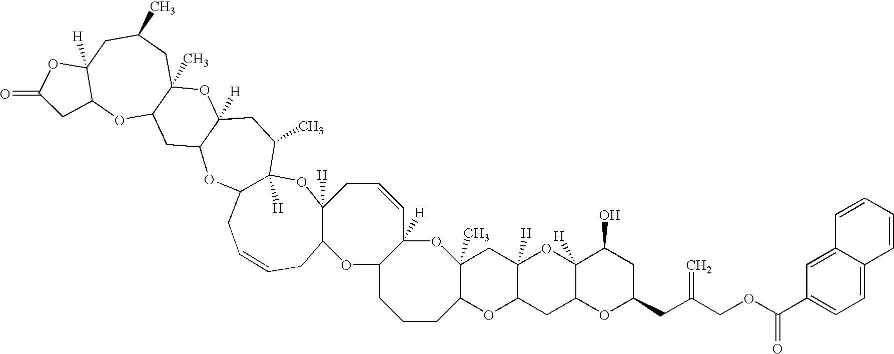

In another preferred embodiment the compound of formula (III) is:

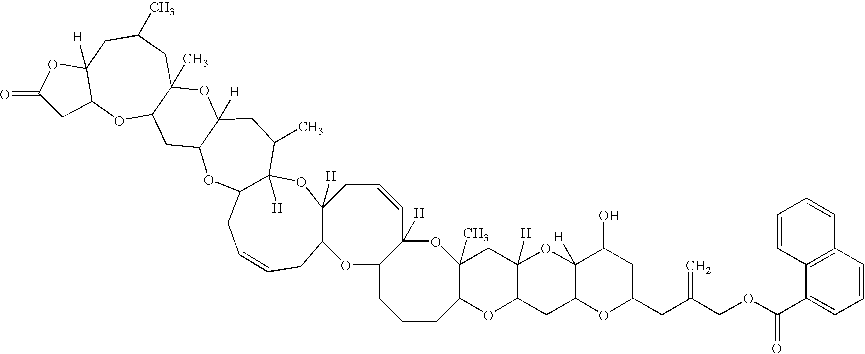

In one aspect of this preferred embodiment, the compound is: 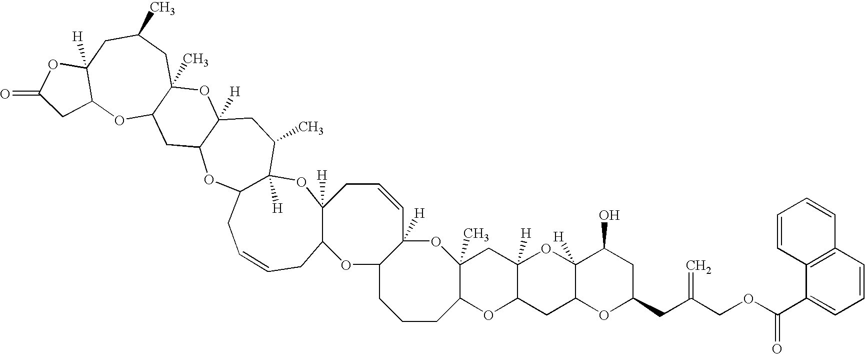
In another preferred embodiment the compound of formula (III) is:

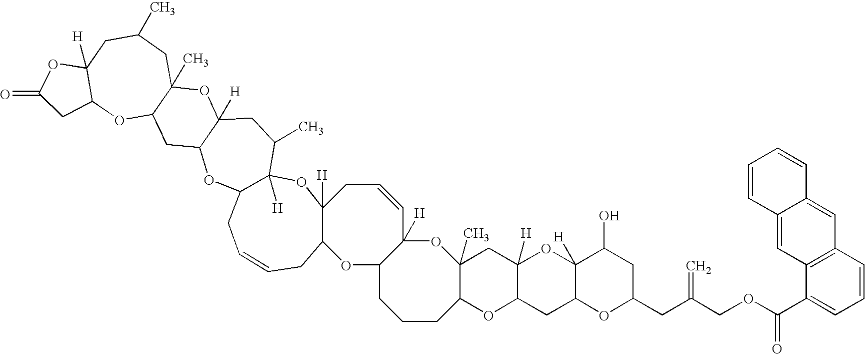

In one aspect of this preferred embodiment, the compound is: 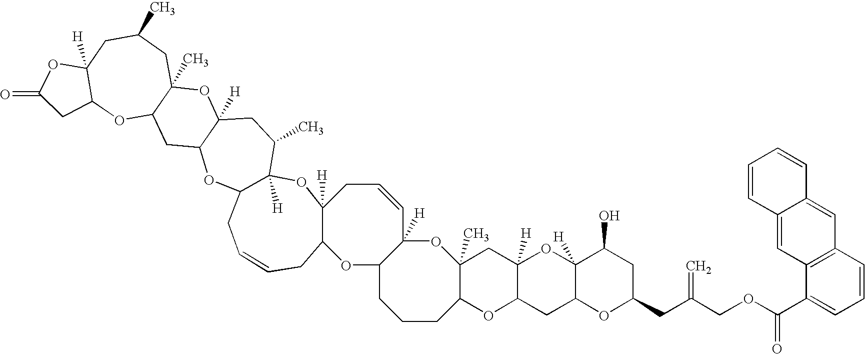
In another preferred embodiment the compound of formula (III) is:

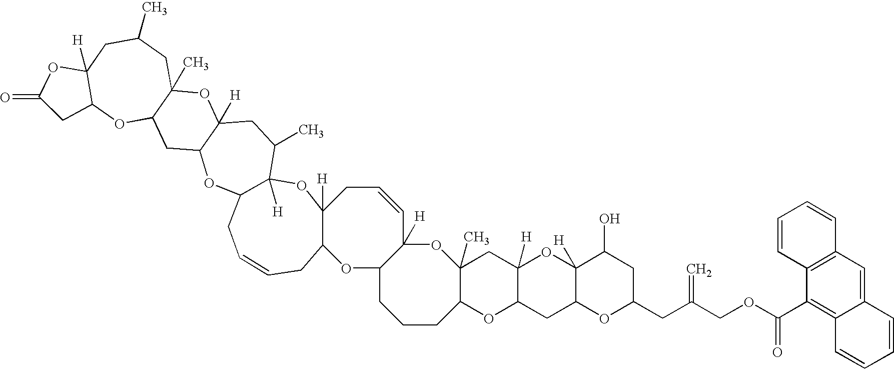

In one aspect of this preferred embodiment, the compound is:

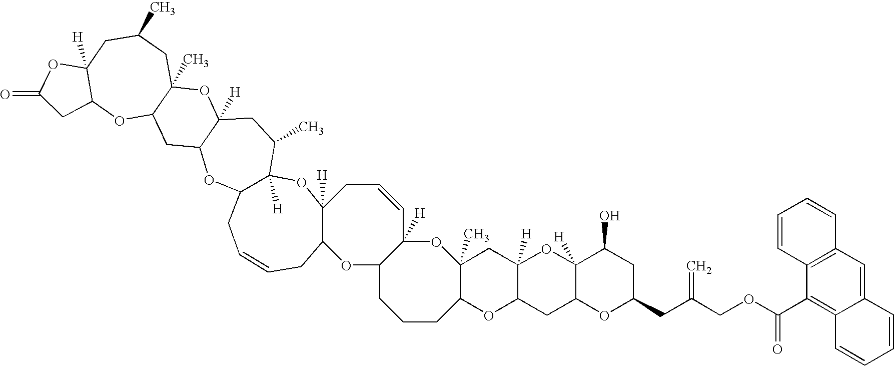

In another preferred embodiment the compound of formula (III) is:

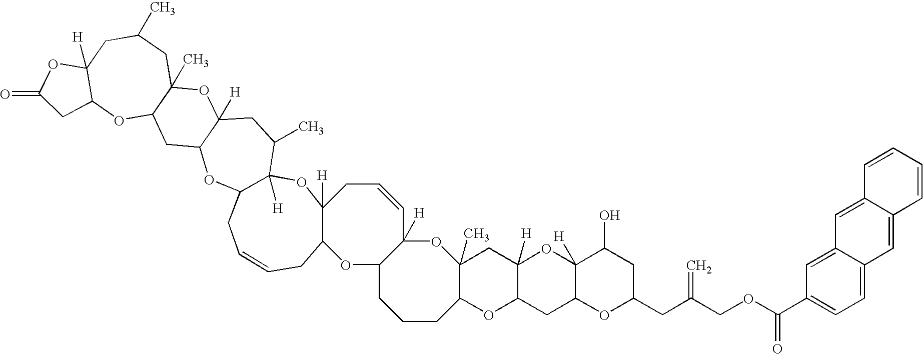

In one aspect of this preferred embodiment, the compound is:

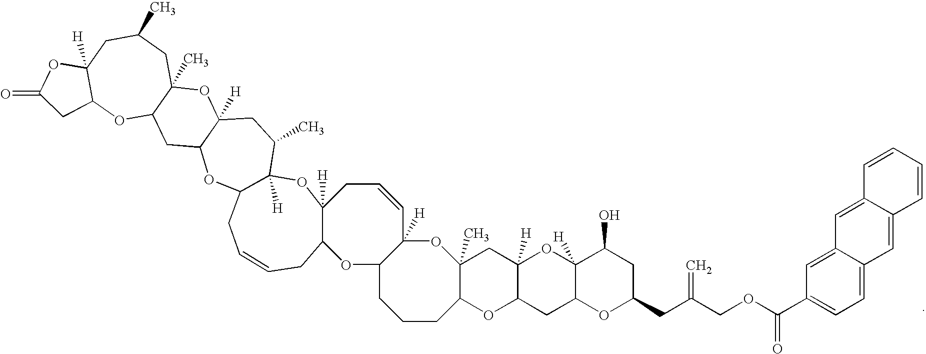

The compounds of Formulas (I)-(IV) may have asymmetric centers and occur as racemates, racemic mixtures and as individual diastereomers, or enantiomers. All isomeric forms are included within the scope of the invention.

In another aspect, the invention relates to pharmaceutical formulations comprising a compound, or pharmaceutically acceptable salt, solvate, hydrate, complex, or combination thereof, of any of Formula (I), (II), (III), or (IV): 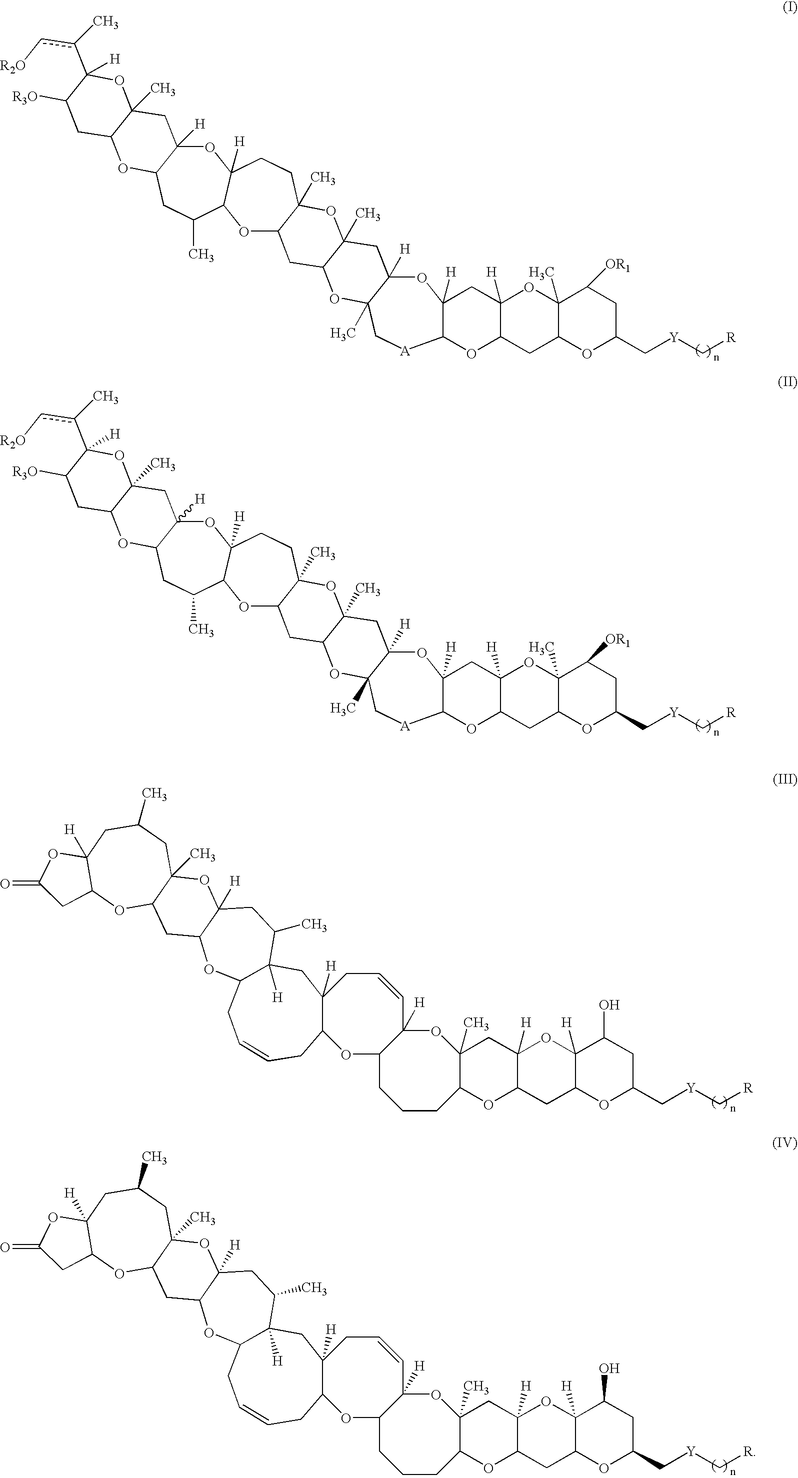
wherein R, R₁, R₂, R₃, R₄, A, n, and Y are as defined herein for each particular formula, with the particular provisos for each particular formula;
and a pharmaceutically acceptable carrier, excipient, solvent, adjuvant or diluent.

In a preferred embodiment of this aspect, the pharmaceutical formulation comprises a compound of Formula (II) or (IV).

In another preferred embodiment, the pharmaceutical formulation comprises a compound of formula:

In another preferred embodiment, the pharmaceutical formulation comprises a compound of formula:

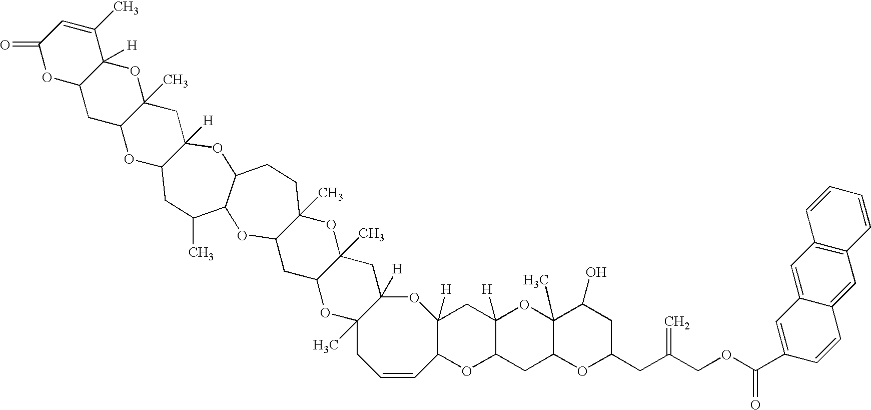

In one aspect of this preferred embodiment, the pharmaceutical formulation comprises a compound of formula:

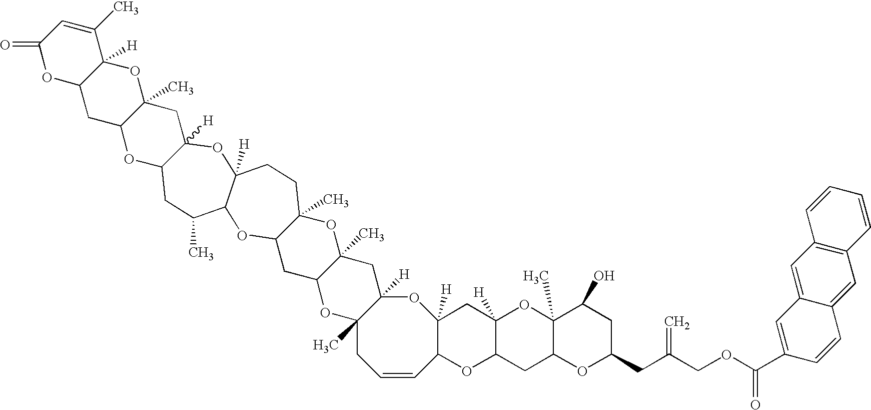

In an other preferred embodiment, the pharmaceutical formulation comprises a compound of formula:

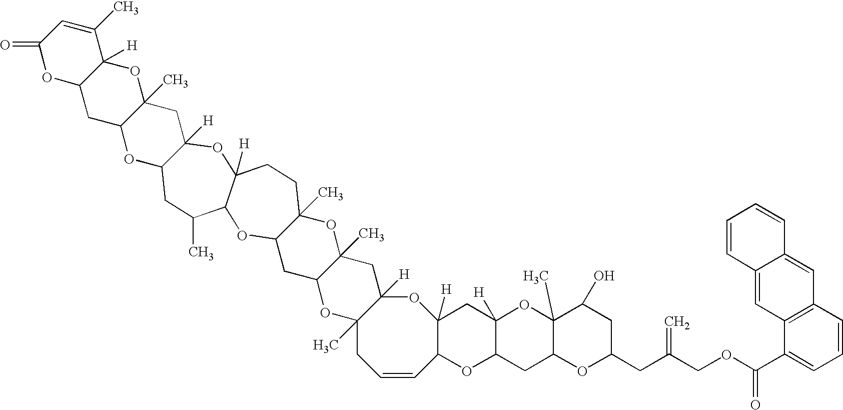

In one aspect of this preferred embodiment, the pharmaceutical formulation comprises a compound of formula:

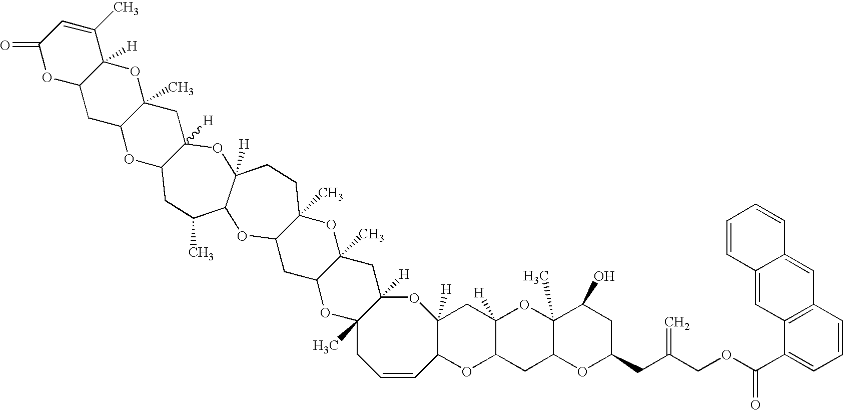

In another preferred embodiment, the pharmaceutical formulation comprises a compound of formula:

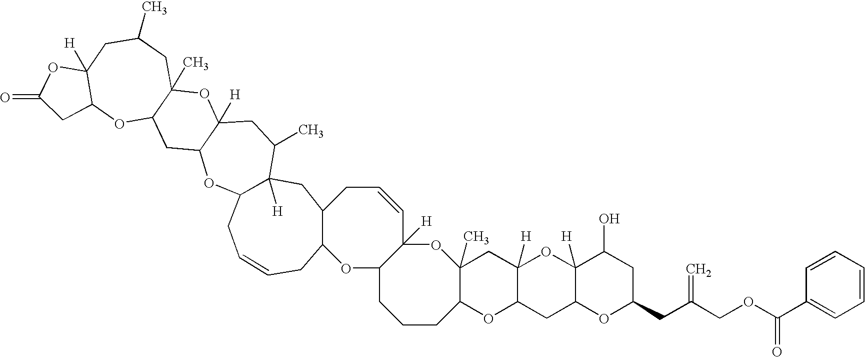

In one aspect of this preferred embodiment, the pharmaceutical formulation comprises a compound of formula:

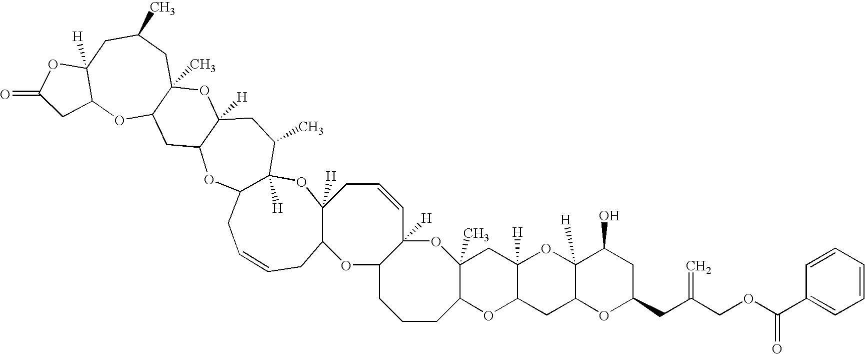

In another preferred embodiment, the pharmaceutical formulation comprises a compound of formula:

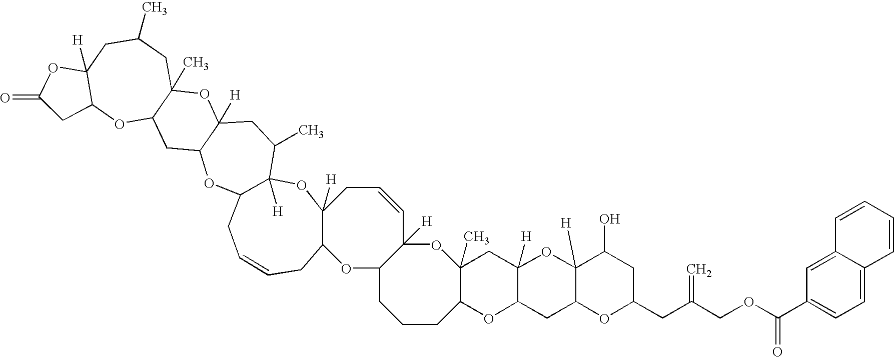

In one aspect of this preferred embodiment, the pharmaceutical formulation comprises a compound of formula:

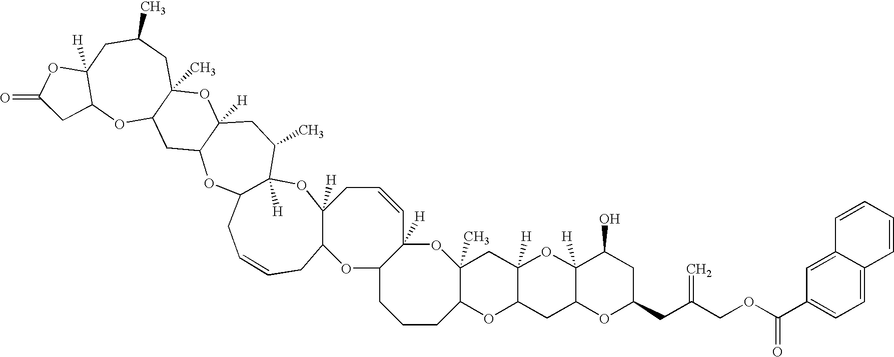

In another preferred embodiment, the pharmaceutical formulation comprises a compound of formula:

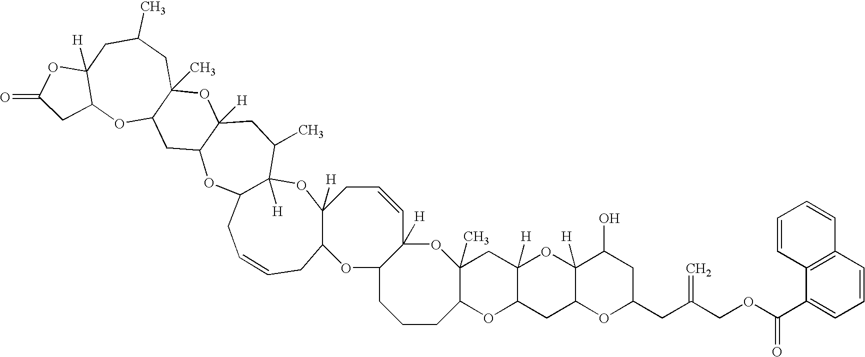

In one aspect of this preferred embodiment, the pharmaceutical formulation comprises a compound of formula:

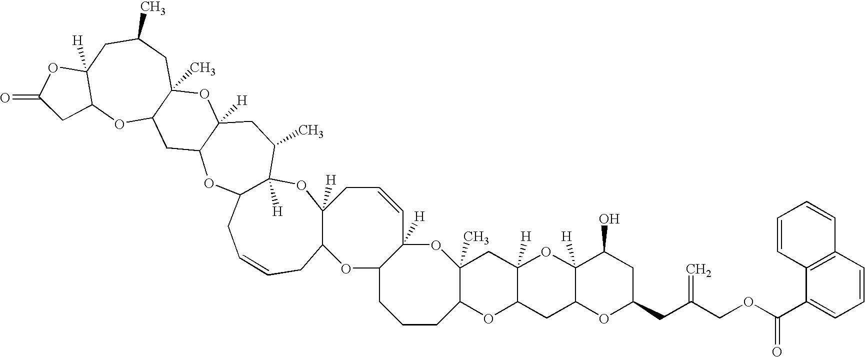

In another preferred embodiment, the pharmaceutical formulation comprises a compound of formula:

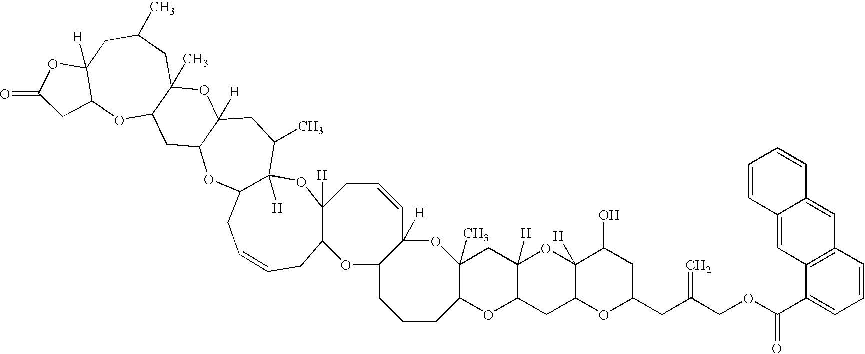

In one aspect of this preferred embodiment, the pharmaceutical formulation comprises a compound of formula:

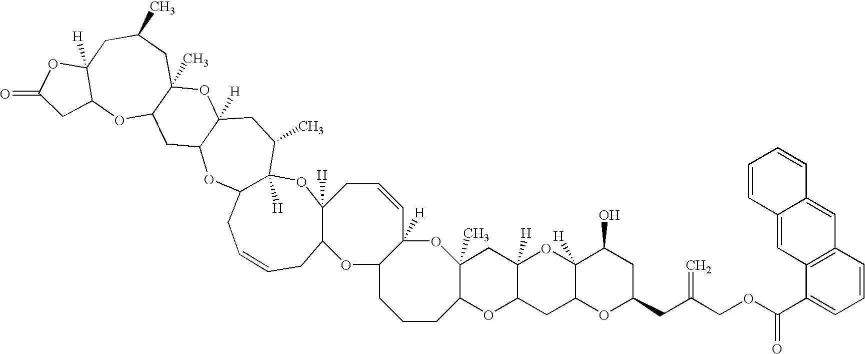

In another preferred embodiment, the pharmaceutical formulation comprises a compound of formula:

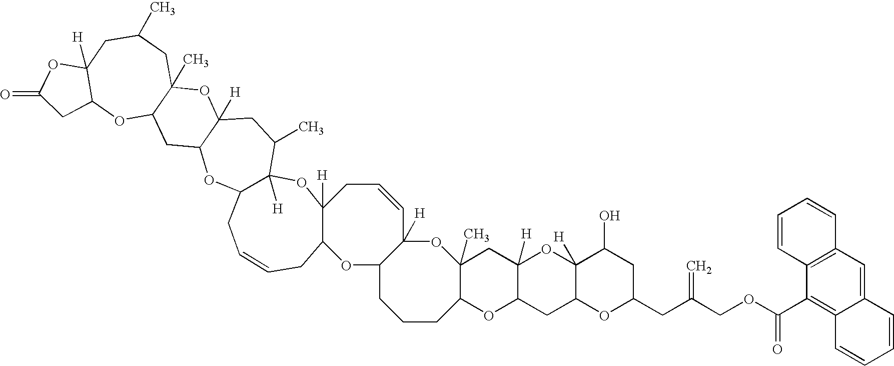

In one aspect of this preferred embodiment, the pharmaceutical formulation comprises a compound of formula:

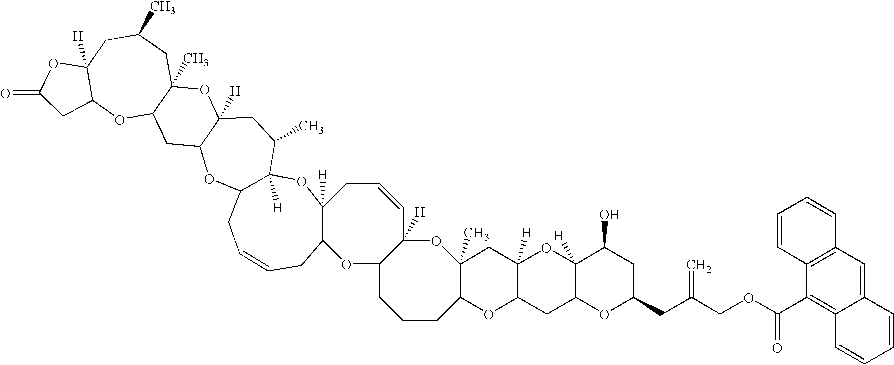

In another preferred embodiment, the pharmaceutical formulation comprises a compound of formula:

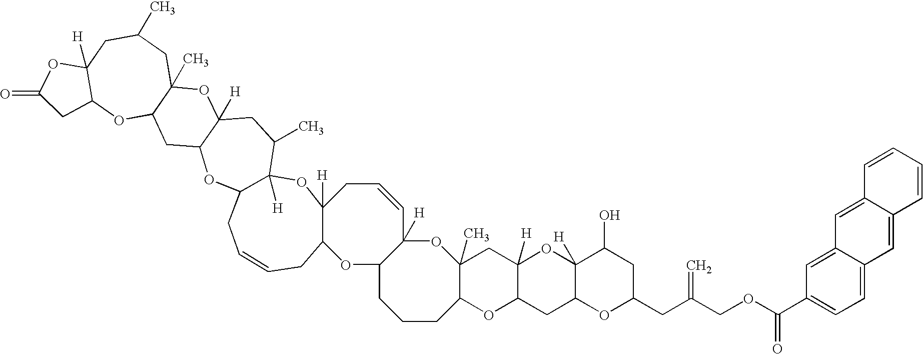

In one aspect of this preferred embodiment, the pharmaceutical formulation comprises a compound of formula:

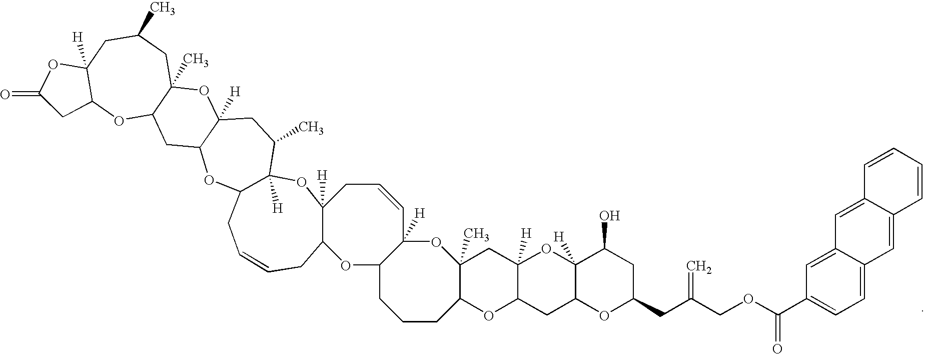

The invention also provides methods for treating neurotoxic shellfish poisoning (NSP) and/or ciguatera fish poisoning (CFP), comprising administering to a subject a compound of the invention, or a pharmaceutically acceptable salt, solvate, hydrate, complex, or combination thereof, in an amount effective to treat NSP and/or CFP. This method of treating NSP and/or CFP can help prevent, treat, reduce the severity of, or delay the onset or progression of symptoms and disease states associated with NSP and/or CFP.

In another aspect, the invention provides methods for treating the symptoms related to NSP and/or CFP, comprising administering to a subject in need of such treatment, a therapeutically effective amount of a compound of the invention, or a pharmaceutically acceptable salt, solvate, hydrate, complex, or combination thereof.

In another embodiment of this aspect, the method can optionally comprise in combination with the compound of Formula (I)-(IV), or pharmaceutically acceptable salts, solvate, hydrate, complex, or combination thereof, an effective amount of a compound known to be useful for the treatment of CFP and/or NSP, such as, for example, mannitol. The methods of the invention can optionally comprise additional therapeutic regimen such as supportive or adjuvant therapy.

In one embodiment of the methods of the invention, the subject is a mammal. In a more preferred embodiment, the mammal is a human.

The methods of the invention employ therapeutically effective amounts: for inhalation, oral, parenteral, sublingual, intranasal, intrathecal, depo, implants, topical, and rectal administration from about 0.1 pg/day to about 100 mg/day. The therapeutically effective amounts will vary according to various parameters including, for example, the route of administration, the distribution of the compound, the metabolism of the compound, the excretion of the compound, the particular therapeutic use, and the physical characteristics of the subject/patient, and are well within the knowledge of those skilled in the art.

In a preferred aspect, the therapeutically effective amounts for oral administration is about 1 mg/day to about 100 mg/day.

In a preferred aspect, the therapeutically effective amounts for parenteral, and depo administration is from about 1 pg/day to about 100 mg/day.

The invention also includes the use of a compound of Formula (I)-(IV), or a pharmaceutically acceptable salt, solvate, hydrate, complex, or combination thereof for the manufacture of a medicament for use in treating a subject who has, or in preventing a subject from developing, NSP and/or CFP and symptoms associated with those poisonings, and who is in need of such treatment.

In one aspect, this use of a compound of formula (I)-(IV) can be employed where the poisoning is neurotoxic shellfish poisoning.

In another aspect, this use of a compound of formula (I)-(IV) can be employed where the poisoning is ciguatera fish poisoning.

In another aspect, this use of a compound of formula (I)-(IV) can be employed where the symptoms are associated with neurotoxic shellfish poisoning.

In another aspect, this use of a compound of formula (I)-(IV) can be employed where the symptoms are associated with ciguatera fish poisoning.

The invention also includes a container kit including a plurality of containers, each container including one or more unit dose of a compound of formula (I)-(IV), or a pharmaceutically acceptable salt thereof.

In an embodiment, this container kit includes each container adapted for oral delivery and includes an inhaler, tablet, gel, or capsule.

In an embodiment, this container kit includes each container adapted for parenteral delivery and includes a depot product, syringe, ampoule, or vial.

In an embodiment, this container kit includes each container adapted for topical delivery and includes a patch, medipad, ointment, or cream.

In each of these kit embodiments, the kits further comprise directions for use of the particular form of delivery.

The compounds of formulas (I)-(IV) can form salts when reacted with appropriate acids or bases. Pharmaceutically acceptable salts are generally preferred over the corresponding compounds of formula (I)-(IV) since they frequently produce compounds that are usually more water soluble, stable and/or more crystalline. Pharmaceutically acceptable salts are any salt which retains the activity of the parent compound and does not impart any deleterious or undesirable effect on the subject to whom it is administered and in the context in which it is administered. Pharmaceutically acceptable salts include acid addition salts of both inorganic and organic acids. Preferred pharmaceutically acceptable salts include salts such as those described by Berge, Bighley, and Monkhouse, J. Pharm. Sci., 1977, 66, 1-19. Such salts may be formed from inorganic and organic acids. Representative examples thereof include maleic, fumaric, benzoic, ascorbic, pamoic, succinic, bismethylenesalicylic, methanesulfonic, ethanedisulfonic, acetic, propionic, tartaric, salicylic, citric, gluconic, aspartic, stearic, palmitic, itaconic, glycolic, p-aminobenzoic, glutamic, benzenesulfonic, hydrochloric, hydrobromic, sulfuric, cyclohexylsulfamic, phosphoric and nitric acids. For other acceptable salts, see Int. J. Pharm., 33, 201-217 (1986). The compounds of formulas (I)-(IV) can also form solvates, hydrates, or complexes, or combinations thereof.

Methods of the Invention

The compounds of the invention, pharmaceutical formulations comprising said compounds, and pharmaceutically acceptable salts thereof, are useful for treating mammals suffering from a disease or condition characterized by at least one symptom of neurotoxic shellfish poisoning or ciguatera fish poisoning, and are useful for helping to prevent or delay the onset of such a condition. The compounds and formulations of the invention are particularly useful for treating, preventing, or slowing the progression of neurological, gastrointestinal, and cardiovascular symptoms of NSP or CFP. When treating or preventing NSP and CFP, and the associated symptoms, the compounds of the invention can either be used individually or in combination, as is best for the subject.

With regard to NSP and CFP, the term “treating” means that compounds of the invention can be used in subjects, preferably human subjects/patients, with existing NSP or CFP. The compounds of the invention will not necessarily cure the subject who has the particular poisoning, but will abate or slow the progression or prevent further progression of the poisoning (and the associated symptoms) thereby providing relief to the individual.

The term “preventing” means that that if the compounds of the invention are administered to those who do not show symptoms of CFP or NSP but who may develop the symptoms, or be at increased risk for CFP or NSP, they will not develop the symptoms of the poisoning. In addition, “preventing” also includes decreasing the severity of the symptoms associated with NSP or CFP, in an individual who may ultimately develop CFP or NSP and the associated symptoms, or would be at risk for CFP or NSP due to the known exposure to brevetoxins and/or ciguatoxin.

In a preferred aspect, the compounds of the invention are useful for slowing the progression of NSP and/or CFP symptoms.

In another preferred aspect, the compounds of the invention are useful for preventing the further progression of NSP and/or CFP symptoms.

In treating or preventing the above poisonings, the compounds of the invention are administered in a therapeutically effective amount. The therapeutically effective amount will vary depending on the particular compound used, the physical characteristics of the subject to be treated, and the route of administration, as is known to those skilled in the art.

In treating a subject displaying any of the diagnosed above conditions a physician may administer a compound of the invention immediately and continue administration indefinitely, as needed.

Dosage Forms and Amounts

The compounds of the invention can be administered orally, parenterally, (IV, IM, depo-IM, SQ, and depo SQ), sublingually, intranasally, by inhalation, intrathecally, topically, vaginally, or rectally. Dosage forms known to those of skill in the art are suitable for delivery of the compounds of the invention.

Compositions are provided that contain therapeutically effective amounts of the compounds of the invention. The compounds are preferably formulated into suitable pharmaceutical preparations such as aerosols, inhalants, tablets, capsules, or elixirs for oral administration or in sterile solutions or suspensions for parenteral administration. Typically the compounds described above are formulated into pharmaceutical compositions using techniques and procedures well known in the art.

About 0.1 pg to about 100 mg of a compound or mixture of compounds of the invention or a physiologically acceptable salt, solvate, hydrate, complex, ester, or combination thereof, is compounded with a physiologically acceptable vehicle, carrier, excipient, binder, preservative, stabilizer, flavor, etc., in a unit dosage form as called for by accepted pharmaceutical practice. The amount of active substance in those compositions or preparations is such that a suitable dosage in the range indicated is obtained. The compositions are preferably formulated in a unit dosage form, each dosage containing from about 0.1 pg to about 100 mg, preferably about 1 pg to about 1 μg for inhalation administration, preferably about 100 ng to about 1 mg for injection/intravenous adiministration, or about 1 mg to about 100 mg for oral administration (e.g., tablets, elixirs, capsules, etc.), of the active ingredient. The term “unit dosage from” refers to physically discrete units suitable as unitary dosages for human subjects and other mammals, each unit containing a predetermined quantity of active material calculated to produce the desired therapeutic effect, in association with a suitable pharmaceutical excipient.

To prepare pharmaceutical compositions, one or more compounds of the invention are mixed with a suitable pharmaceutically acceptable carrier. Upon mixing or addition of the compound(s), the resulting mixture may be a solution, suspension, emulsion, or the like. Liposomal suspensions may also be suitable as pharmaceutically acceptable carriers. These may be prepared according to methods known to those skilled in the art. The form of the resulting mixture depends upon a number of factors, including the intended mode of administration and the solubility of the compound in the selected carrier or vehicle. The effective concentration is sufficient for lessening or ameliorating at least one symptom of the disease, disorder, or condition treated and may be empirically determined.

Pharmaceutical carriers or vehicles suitable for administration of the compounds provided herein include any such carriers known to those skilled in the art to be suitable for the particular mode of administration. In addition, the active materials can also be mixed or blended with other active materials that do not impair the desired action, or with materials that supplement the desired action, or have another action. The compounds may be formulated as the sole pharmaceutically active ingredient in the composition or may be combined with other active ingredients.

Where the compounds exhibit insufficient solubility, methods for solubilizing may be used. Such methods are known and include, but are not limited to, using cosolvents such as dimethylsulfoxide (DMSO), using surfactants such as Tween®, and dissolution in aqueous sodium bicarbonate. Derivatives of the compounds, such as salts, solvates, hydrates, complexes, or prodrugs may also be used in formulating effective pharmaceutical compositions.

The concentration of the compound is effective for delivery of an amount upon administration that lessens or ameliorates at least one symptom of the poisoning or disorder for which the compound is administered. Typically, the compositions are formulated for single dosage administration.

The compounds of the invention may be prepared with carriers that protect them against rapid elimination from the body, such as time-release formulations or coatings. Such carriers include controlled release formulations, such as, but not limited to, microencapsulated delivery systems. The active compound is included in the pharmaceutically acceptable carrier in an amount sufficient to exert a therapeutically useful effect in the absence of undesirable side effects on the subject treated. The therapeutically effective concentration may be determined empirically by testing the compounds in known in vitro and in vivo model systems for the treated disorder.

The compounds and compositions of the invention can be enclosed in multiple or single dose containers. The enclosed compounds and compositions can be provided in kits, for example, including component parts that can be assembled for use. For example, a compound inhibitor in lyophilized form and a suitable diluent may be provided as separated components for combination prior to use. A kit may include a compound inhibitor and a second therapeutic agent for co-administration. The inhibitor and second therapeutic agent may be provided as separate component parts. A kit may include a plurality of containers, each container holding one or more unit dose of the compound of the invention. The containers are preferably adapted for the desired mode of administration, including, but not limited to tablets, gel capsules, sustained-release capsules, and the like for oral administration; depot products, pre-filled syringes, ampoules, vials, and the like for parenteral administration; and patches, medipads, creams, and the like for topical administration.

The concentration of active compound in the drug composition will depend on absorption, route of administration, metabolism, inactivation, and excretion rates of the active compound, the dosage schedule, and amount administered as well as other factors known to those of skill in the art.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

If oral, non-inhalation administration is desired, the compound should be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

Oral compositions will generally include an inert diluent or an edible carrier and may be compressed into tablets or enclosed in gelatin capsules. For the purpose of oral therapeutic administration, the active compound or compounds can be incorporated with excipients and used in the form of tablets, capsules, or troches. Pharmaceutically compatible binding agents and adjuvant materials can be included as part of the composition.

The tablets, pills, capsules, troches, and the like can contain any of the following ingredients or compounds of a similar nature: a binder such as, but not limited to, gum tragacanth, acacia, corn starch, or gelatin; an excipient such as microcrystalline cellulose, starch, or lactose; a disintegrating agent such as, but not limited to, alginic acid and corn starch; a lubricant such as, but not limited to, magnesium stearate; a gildant, such as, but not limited to, colloidal silicon dioxide; a sweetening agent such as sucrose or saccharin; and a flavoring agent such as peppermint, methyl salicylate, or fruit flavoring.

When the dosage unit form is a capsule, it can contain, in addition to material of the above type, a liquid carrier such as a fatty oil. In addition, dosage unit forms can contain various other materials, which modify the physical form of the dosage unit, for example, coatings of sugar and other enteric agents. The compounds can also be administered as a component of an elixir, suspension, syrup, wafer, chewing gum or the like. A syrup may contain, in addition to the active compounds, sucrose as a sweetening agent and certain preservatives, dyes and colorings, and flavors.

The active materials can also be mixed or blended with other active materials that do not impair the desired action, or with materials that supplement the desired action.

Solutions or suspensions used for parenteral, intradermal, subcutaneous, or topical application can include any of the following components: a sterile diluent such as water for injection, saline solution, fixed oil, a naturally occurring vegetable oil such as sesame oil, coconut oil, peanut oil, cottonseed oil, and the like, or a synthetic fatty vehicle such as ethyl oleate, and the like, polyethylene glycol, glycerine, propylene glycol, or other synthetic solvent; antimicrobial agents such as benzyl alcohol and methyl parabens; antioxidants such as ascorbic acid and sodium bisulfite; chelating agents such as ethylenediaminetetraacetic acid (EDTA); buffers such as acetates, citrates, and phosphates; and agents for the adjustment of tonicity such as sodium chloride and dextrose. Parenteral preparations can be enclosed in ampoules, disposable syringes, or multiple dose vials made of glass, plastic, or other suitable material. Buffers, preservatives, antioxidants, and the like can be incorporated as required.

Where administered intravenously, suitable carriers include physiological saline, phosphate buffered saline (PBS), and solutions containing thickening and solubilizing agents such as glucose, polyethylene glycol, polypropyleneglycol, and mixtures thereof. Liposomal suspensions including tissue-targeted liposomes may also be suitable as pharmaceutically acceptable carriers. These may be prepared according to methods known for example, as described in U.S. Pat. No. 4,522,811.

The active compounds may be prepared with carriers that protect the compound against rapid elimination from the body, such as time-release formulations or coatings. Such carriers include controlled release formulations, such as, but not limited to, implants and microencapsulated delivery systems, and biodegradable, biocompatible polymers such as collagen, ethylene vinyl acetate, polyanhydrides, polyglycolic acid, polyorthoesters, polylactic acid, and the like. Methods for preparation of such formulations are known to those skilled in the art.

The compounds of the invention can be administered by inhalation, orally or intranasally, parenterally (IV, IM, depo-IM, SQ, and depo-SQ), sublingually, intrathecally, topically, or rectally. Dosage forms known to those skilled in the art are suitable for delivery of the compounds of the invention.

Compounds of the invention may be administered enterally or parenterally. When administered orally, compounds of the invention can be administered in usual dosage forms for oral administration as is well known to those skilled in the art. These dosage forms include the usual solid unit dosage forms of tablets and capsules as well as liquid dosage forms such as solutions, suspensions, and elixirs. When the solid dosage forms are used, it is preferred that they be of the sustained release type so that the compounds of the invention need to be administered only once or twice daily.

The oral dosage forms are administered to the subject 1, 2, 3, or 4, or as needed, times daily. It is preferred that the compounds of the invention be administered either three or fewer times, more preferably once or twice daily. Hence, it is preferred that the compounds of the invention be administered in oral dosage form. It is preferred that whatever oral dosage form is used, that it be designed so as to protect the compounds of the invention from the acidic environment of the stomach. Enteric coated tablets are well known to those skilled in the art. In addition, capsules filled with small spheres each coated to protect from the acidic stomach, are also well known to those skilled in the art.

In a preferred embodiment, the compounds of the invention are administered in an inhalant form.

As noted above, depending on whether asymmetric carbon atoms are present, the compounds of the invention can be present as mixtures of isomers, as racemates, or in the form of pure isomers.

Salts of compounds are preferably the pharmaceutically acceptable or non-toxic salts of compounds of formula I. For isolation and purification purposes it is also possible to use pharmaceutically unacceptable salts.

Synthesis of Compounds

Various synthetic methodologies can be used to make compounds of the invention; certain of the brevetoxins are suitable starting materials. Suitable methodologies are known in the art. Representative synthetic procedures for preparing compounds of the invention from such starting materials are disclosed in, e.g., Mende, T. J., et al., Tetr. Lett., 1990; 31(37):5307-5310; Trainer, V. L., et al., Molec. Pharm., 1991; 40(6):988-994; Keck, G. E., et al., Tetrahedron Lett., 1987, 28:139-142; Alvarez, E., et al., Chem. Rev., 1995, 95:1953-1980; Rein, et al., 1994: (a) J. Org Chem., 59:2107-2113; (b) J. Org. Chem. 59:2101-2106. Each of these references is incorporated herein by reference in its entirety. Those skilled in the art will appreciate that minor modifications can be made to the particular procedures to arrive at compounds of the invention.

The following examples serve merely to illustrate the invention and should not be viewed to limit the invention in scope or spirit.

### EXAMPLES

General. All solvents used were HPLC grade. Brevetoxins were purified from laboratory cultures of the algae Karenia brevis (also called Ptychodiscus brevis and Gymnodinium breve) by a combination of chloroform/methanol extraction and TLC. Brevetoxin can be isolated and purified from native sources, such as K. brevis, or other red tide organisms. Suitable purification methodologies are well known in the art. Preferably, brevetoxins are extracted from K. brevis cultures. This algae is available from the Provasoli Guillard National Center for Culture of Marine Phytoplankton, West Boothbay Harbor, Me., as strain number CCMP718. In addition, the synthesis of Brevetoxin B has been reported: J. Am. Chem. Soc., 117, 1171 (K. C. Nicolaou et al., 1995).

Starting materials (PbTx-2, -3, and -9) and products were routinely purified by reversed phase HPLC (85% isocratic methanol) using a Microsorb-MV, C-18 column (5 um, 25-cm bed) and monitored by UV at 215 or 195 nM and/or refractive index. Proton NMR spectra were recorded in CDCl₃ (CHCl₃ internal standard) at 400 MHz. Mass spectra were run in either DCI or FAB mode. High-resolution mass spectra were obtained from the mass spectrometry facility at the University of California, Riverside.

Synthesis of Brevetoxin Derivatives

A tenfold excess (relative to PbTx-3) of carbonyl diimidazole and the corresponding acid (benzoic, α-naphthoic or β-naphthoic) are combined under nitrogen, at room temperature, in dry benzene. The solution was stirred for 30 min and then added to PbTx-3 in a 5 ml reaction vial. The reaction vial was sealed and the mixture was stirred overnight at 80° C. The reaction mixture was washed with an equal volume (3×) of saturated sodium bicarbonate, an equal volume (3×) of 10% HCl and evaporated under vacuum. The residue was purified using HPLC.

Benzoyl-PbTx-3 (1). Diagnostic peaks in the ¹H NMR include 87.44 (2H, t, J=7.2 Hz), 7.56 (1H, t, J=7.2 Hz), 8.061 (2H, d, J=8.4 Hz), 4.81 (2H, dd, J=5.2 Hz) (C42). The C42 methylene is typically shifted downfield from its position in PbTx-3, and these diastereotopic protons are split into a doublet of doublets in the esters, whereas they appear as a singlet in PbTx-3. DCI MS (NH₃): 1002 (M+1). HRMS (FAB): calc'd for CS7H,,O,S (MH⁺), calc'd 1001.5262, found 1001.5287.

α-Naphthoyl-PbTx-3 (2). Diagnostic peaks in the ¹H NMR include 88.93 (1H, d, J=8.8 Hz), 8.23 (1H, d, J=8.8 Hz), 8.03 (1H, d, J=8.8 Hz), 7.83 (1H, d, J=8.8 Hz), 7.64 (1H, t, J=8.8 Hz), 7.44 (2H, m), 4.92 (2H, dd, J=5.2 Hz) DCI MS (NH₃): 1052 (M+1). HRMS (FAB): calc'd for C6iH,90,5 (MH⁺), calc'd 1051.5419, found 1051.5367.

β-Naphthoyl-Pb7x-3 (3). Diagnostic peaks in the ¹H NMR include 88.64 (1H, s), 7.95 (3H, m), 7.58 (3H, m) 4.89 (1H, s).

### Example 1

Oxidation of C-42 of PbTx-2 to Ester (1). PbTx-2 (4.8 mg, 5.37 uM) was oxidized with activated Mn0₂, to the corresponding methyl ester 1, via the cyanohydrin, according to the procedure described by Corey. The reaction mixture was filtered through Celite® and concentrated in vacuo. The residue was taken up in water (15 mL) and extracted with ether (3×15 mL). The ether phase was evaporated in vacuo and the residue purified by HPLC to yield 3.624 mg (73%) of the desired product. DCI MS (NH₃): 925 (M+1), 942 (M+NH4), 906 (M−H₂O).

### Example 2

Hydrolysis of Methyl Ester 1 To Provide Carboxylic Acid (2). Methyl ester 1 (3.759 mg, 4.068 uM) was dissolved in 2 mL of THF/H₂O (50:50). An aqueous solution of KOH (0.4 mL, 10 mg/mL) was added, and the reaction mixture was stirred at ambient temperature for 2 days. Water (1 mL) was added and the mixture extracted with ether (3×2 mL). The aqueous phase was acidified with 10% HCl and extracted with ethyl acetate (3×2 mL) and the organic phase evaporated in vacuo. This residue consisted of a mixture of two products. On the basis of NMR data, these two products appear to be the C-42 carboxylic acid with an intact A-ring lactone and the C-42 carboxylic acid with a hydrolyzed A-ring lactone. The residue was taken up in THF (1 mL), and a catalytic amount of p-toluenesulfonic acid was added. The mixture was stirred for 1 h and then evaporated in vacuo. The residue was taken up in water (1 mL) and extracted with ethyl acetate (3×2 mL). Evaporation of the solvent in vacuo yielded 3.469 mg (93%) of the crude product.

Purification of the residue by HPLC provided 1.019 mg (28%) of 2. IHNMR. DCI MS(NH3): 911 (M+1), 929 (M+NH4), 892 (M−H₂O).

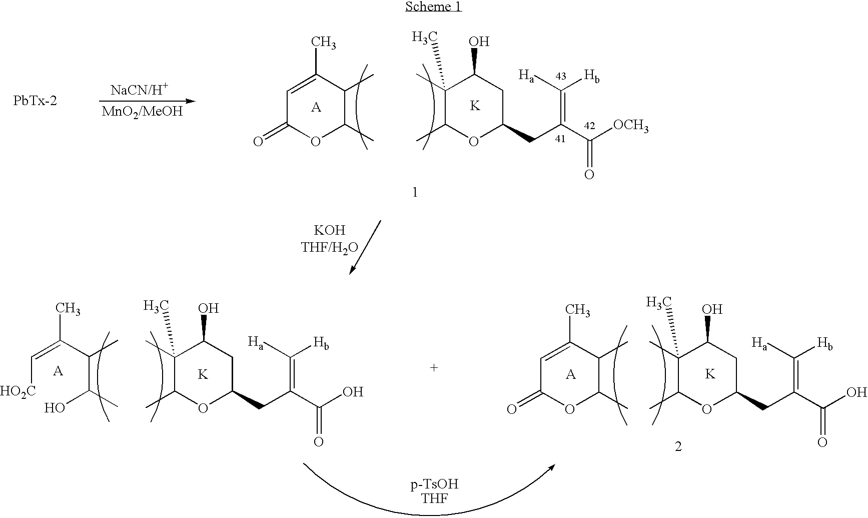

### Example 3

Reduction of the C-2, C-3 Double Bond of PbTx-3 to Provide (3). Following the procedure of Hudlicky, PbTx-3 (4.00 mg, 4.46 uM) and Mg (approximately 200 mg, 99.98% from Timminco metals, Haley, Ontario, Canada) were dried in vacuo over P₂O₆ for 18 h. Methanol (3 mL freshly distilled from CaH₂) was added and the mixture stirred at room temperature (cooling in an ice bath was required) under nitrogen for 2 h. HCl (10%, 10 mL) was added to dissolve the magnesium methoxide and the remaining magnesium. The reaction mixture was concentrated to approximately 5 mL and extracted with ether (3×15 mL). The ether phase was evaporated in vacuo and the residue purified by HPLC. The isolated material consisted of the desired product with the C-2, C-3 double bond reduced and a second product in which the double bond was reduced and the lactone opened to the methyl ester, as evidenced by mass spectral and NMR data. This mixture was taken up in THF, and a small amount of p-TsOH acid was added. The mixture was stirred for 1 h and then evaporated in vacuo. The residue was taken up in water (1 mL) and extracted with ethyl acetate (3×2 mL). Evaporation in vacuo yielded 3.256 mg (81%) of 3. DCI MS(NH3): 899 (M+1), 916 (M+NH,,), 880 (M−H₂O). HRMS (DCI): calcd for Cr,oH74014 (MH⁺) 899.5156, found 899.5128.

### Example 4

Reduction of the C-2, C-3 Double Bond of PbTx-9 to Provide (4). PbTx-9 (7.49 mg, 8.34 AM) was reduced according to the procedure described for the preparation of 3 to yield 1.645 mg (22%) of 4. The ¹H NMR spectrum is shown in the supplementary material. DCI MS(NH3): 901 (M+1), 918 (M+NH4), 882 (M−H,O. HRMS (DCI): calcd for C6pHqg014 (MH+) 901.5313, found 901.5323.

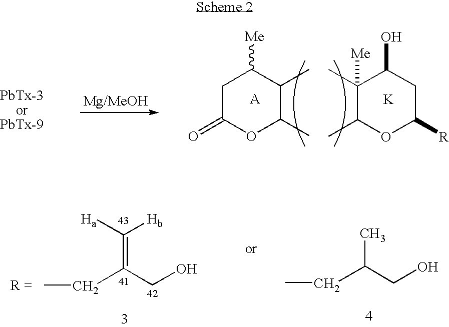

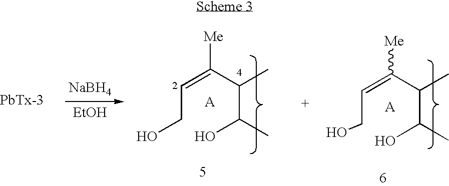

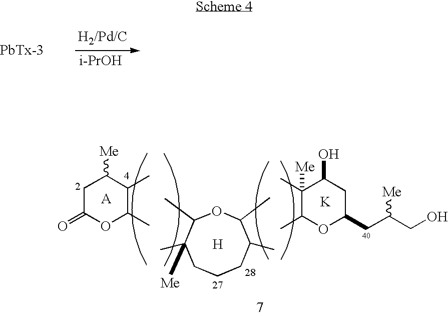

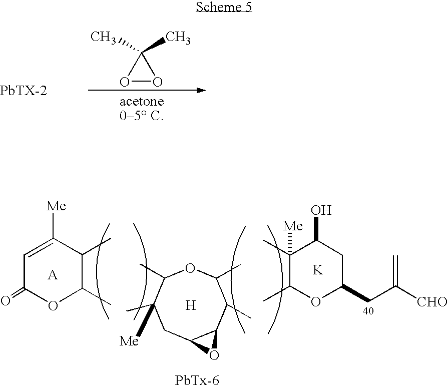

### Example 5

Sodium Borohydride Reduction of PbTx-3 to Form (5) and (6).

PbTx-3 (3.451 mg, 3.85 uM) was dissolved in 2.5 mL of EtOH. A large excess of NaBH₄ (5 mg) was added in one portion. The reaction mixture was stirred at ambient temperature for 18 h. The excess NaBH₄ was decomposed by the careful addition of 10% HCl. The reaction mixture was concentrated in vacuo to 1 mL and extracted with CH₂Cl₂ (3×2 mL). The combined organic phases were then evaporated to dryness, and the residue was purified by HPLC. Two peaks were collected from the HPLC. The first peak was the minor product 5, 0.755 mg (22%), and the second peak was the major product 6,1.042 mg (30%). Compound 5. DCI MS(NH3): 880, 729. FAB MS (m-nitrobenzyl alcohol matrix): 901 (M+1). HRMS (FAB): calcd for C6pHyg014 (MH⁺) 901.5313, found 901.5324. Compound 6. FAB MS (m-nitrobenzyl alcohol matrix): 903 (M+1), 766, 731. HRMS (FAB): calcd for CrOH78014 (MH⁺) 903.5470, found 903.5418.

### Example 6

Catalytic Reduction of PbTx-3 to Yield (7). PbTx-3 (1.8 mg, 2.00 wM) was dissolved in i-PrOH (1 mL). Acetic acid (50 AL) and a catalytic amount of 10% Pd on activated carbon were added. The reaction mixture was stirred at ambient temperature under an atmosphere of H₂ for 24 h. The suspension was filtered through Celite® and concentrated in vacuo to provide 0.986 mg (54%) of 7 which was not purified further. DCI MS (NH3): 903 (M+1), 920 (M+NH4), 894 (M−H₂O). HRMS (DCI): calcd for CrOH.8014 (MH⁺) 903.5470, found 903.5444.

### Example 7

Epoxidation of the C-27, C-28 Double Bond of PbTx-2 To Provide PbTx-6. Dimethyldioxirane was generated in a distillation apparatus, connected to a dry ice condenser, according to the procedure described by Adam17 for a small-scale preparation. The receiving flask was charged with PbTx-2 (2.33 mg) in 5.0 mL of acetone and was cooled in an ice/salt bath. The reaction was monitored by HPLC. When all of the PbTx-2 was consumed, the acetone was evaporated in vacuo, and the residue taken up in 1.0 mL of methanol and purified by HPLC to provide 2.25 mg (95%) of PbTx-6. DCI MS (NH₃): 911 (M+1), 928 (M+NH₄), 893 (M−H₂O). 1H and ¹³C NMR were identical to that reported by Shimizu.

### Example 8

Evaluation of Specific Receptor Binding

A. Preparation of [³H]PbTx-3. Tritiated PbTx-3 was prepared at a specific activity of 12-15 Ci/mmol by reductive tritiation of PbTx-2 using cerium chloride-sodium borotritiide. Specific activity was calculated using HPLC against toxin standards and liquid scintillation counting standardized to a tritium quench curve.

B. Synaptosome Binding Experiments. Synaptosomes were prepared by the method described by Dodd et al. (Brain Res. 1981 Dec. 7;226(1-2):107-18; J. Neurochem. 1983 March;40(3):608-14) from male Sprague-Dawley rats (200-250 g). Total binding was measured using a rapid centrifugation technique described previously. Nonspecific binding was measured in identical incubation tubes by adding 10 uM unlabeled PbTx-3 to completely displace all specifically bound radioactive toxin (radioactivity that cannot be displaced by excess ligand is nonspecific).

[image]

Specific binding was calculated by subtracting nonspecific binding from total binding. All binding experiments were performed in standard binding medium (SBM) consisting of 50 mM HEPES (pH 7.4), 130 mM choline chloride, 5.5 mM glucose, 0.8 mM magnesium sulfate, 5.4 mM potassium chloride, 1 mg/mL of bovine serum albumin, and 0.01% Emulphor EL-620, a nonionic detergent used as an emulsifier. The latter was required to solubilize the high concentration of competitor toxins. A suspension of synaptosomes (40-80 μg of total protein in 100 pL of SBM) was added to microfuge tubes containing [aH]PbTx-3 (1.78 nM) and competitor (12 different concentrations ranging from 0.5 to 25 uM). Binding is measured using traditional receptor binding liquid scintillation counting of membrane pellets. Inhibition of binding of tritiated brevetoxin to site 5 is shown below for PbTx-1 (X), PbTx-2 (▴), PbTx-3 (▪), PbTx-6 (♦), and PbTx-9 (●).

The invention has been described with reference to various specific and preferred embodiments and techniques. However, it should be understood that many variations and modifications may be made while remaining within the spirit and scope of the invention.

1. A method of treating neurotoxic shellfish poisoning in a mammal comprising administering to a subject in need of such treatment a therapeutically effective amount of a compound of formula (I): 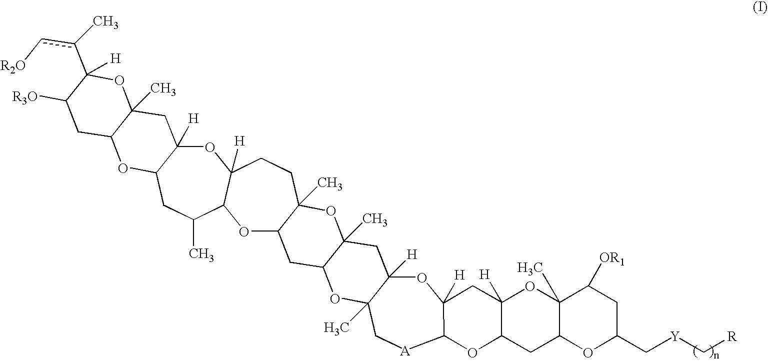
wherein
A is 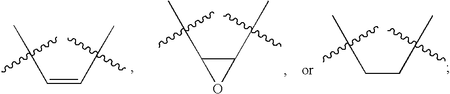
R is C₁-C₆ alkyl, C₂-C₆ alkenyl, C₁-C₆ alkyl ester, C₂-C₆ alkenyl ester, amino, amido, aryl ester, cycloalkyl ester, cycloalkenyl ester, purinyl, pyrimidines, aryl, heterocyclyl, or heteroaryl;
R₁ is H or —(CO)CH₃; and
R₂ and R₃ at each occurrence are independently —CH₂(CO)CH₃, —CH₂ (CO)CH₂CH₃, —CH₂ (CO)CH(CH₃)₂, —CH₂ (CO)CH₂CH₂CH₃, —CH₂ (CO)CH(CH₃) CH₂CH₃, or —CH₂(CO)CH₂CH(CH₃)₂,
or OR₂ and OR₃ can be taken together to form a six membered ring of the formula (Ia) 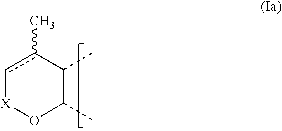
wherein X is C═O or CH(CH₃);
wherein the bracketed-dashed bonds indicate attachment to backbone;
Y is C═CH₂, C═O, CHCH₃, or CH₂;
n is 1 or 0,
or a pharmaceutically acceptable salt, solvate, hydrate, amide, ester, complex, or combination thereof.

2. A method according to claim 1, where the compound has the formula II:

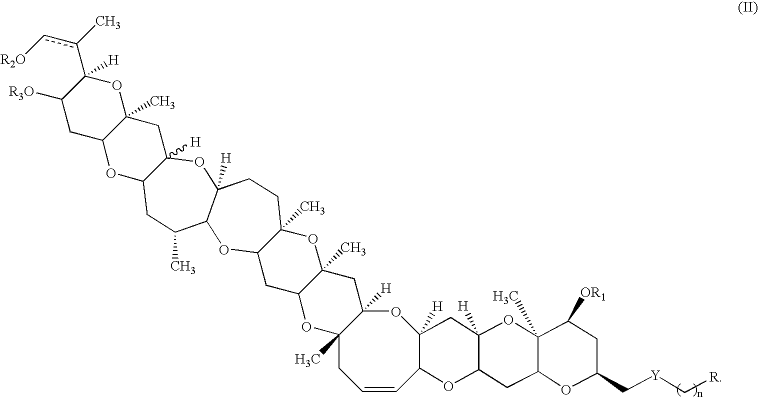

3. A method of treating neurotoxic shellfish poisoning in a subject comprising administering to a subject in need of such treatment a therapeutically effective amount of a compound of formula (III): 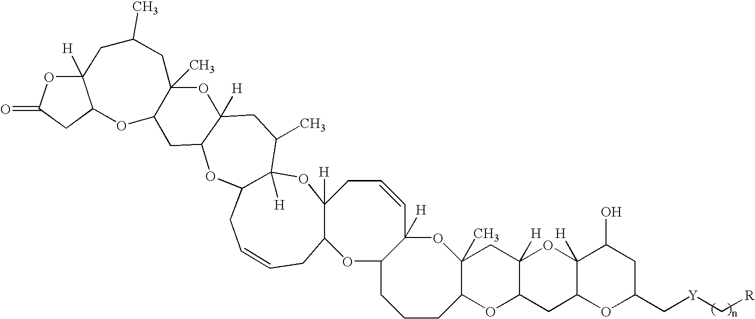
wherein
R is H, OH, halogen, C₁-C₆ lower alkyl, C₁-C₆ alkyl esters, C₂-C₆ alkenyl ester, amino, amido, aldehydo, aryl ester, cycloalkyl ester, cycloalkenyl ester, purine, pyrimidine, aryl, heterocyclyl, or heteroaryl;
Y is C═O, C═CH₂, CHCH₃ or CH₂; and
n is 1 or 0,
or a pharmaceutically acceptable salt, solvate, hydrate, amide, ester, complex, or combination thereof.

4. A method according to claim 3, where the compound has the formula (IV):

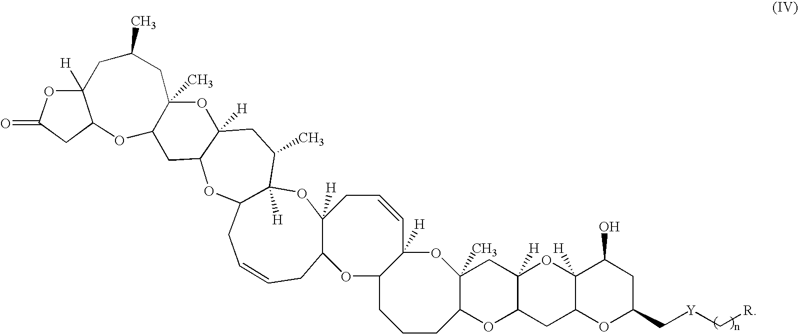

5. A method according to claim 1, wherein the therapeutically effective amount is administered in a dosage of between about 0.1 pg to about 100 mg per day.

6. A method according to claim 1, wherein the therapeutically effective amount comprises a dosage of between about 0.1 pg to about 1.0 μg per day.

7. A method according to claim 1, wherein the therapeutically effective amount of the compound is administered by inhalation.

8. A method according to claim 1, wherein the therapeutically effective amount of the compound is administered orally.

9. A method according to claim 1, where the compound is administered as a pharmaceutical formulation comprising the compound, or at least one pharmaceutically acceptable salt, ester, amide, solvate, hydrate, complex, or combination thereof, and at least one pharmaceutically acceptable carrier, excipient, solvent, diluent, or adjuvant.

10. A method according to claim 3, wherein the compound is administered as a pharmaceutical formulation comprising the compound, or at least one pharmaceutically acceptable salt, solvate, ester, amide, hydrate, complex, or combination thereof, and at least one pharmaceutically acceptable carrier, excipient, solvent, diluent, or adjuvant.

11. A method according to claim 3, wherein the therapeutically effective amount comprises a dosage of between about 0.1 pg to about 100 mg per day.

12. A method according to claim 3, wherein the therapeutically effective amount comprises a dosage of between about 0.1 pg to about 1.0 μg per day.

13. A method according to claim 3, wherein the therapeutically effective amount of the compound is administered by inhalation.

14. A method according to claim 3, wherein the therapeutically effective amount of the compound is administered orally.

15. A method of treating ciguatera fish poisoning in a subject comprising administering to a subject in need of such treatment a therapeutically effective amount of a compound of formula (I): 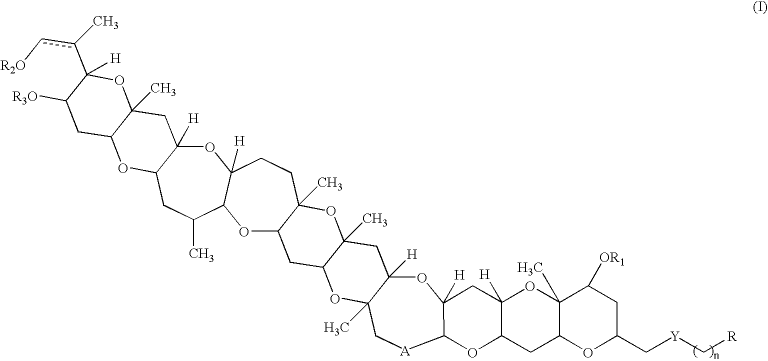
wherein
A is 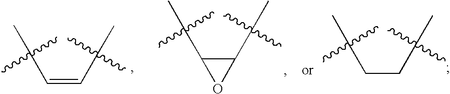
R is C₁-C₆ alkyl, C₂-C₆ alkenyl, C₁-C₆ alkyl esters, C₂-C₆ alkenyl ester, amino, amido, aryl ester, cycloalkyl ester, cycloalkenyl ester, purinyl, pyrimidinyl, heterocyclyl, aryl or heteroaryl;
R₁ is H or —(CO)CH₃; and
R₂ and R₃ at each occurrence are independently —CH₂(CO)CH₃, —CH₂ (CO)CH₂CH₃, —CH₂ (CO)CH(CH₃) 2, —CH₂ (CO)CH₂CH₂CH₃, —CH₂ (CO)CH(CH₃)CH₂CH₃, or —CH₂(CO)CH₂CH(CH₃)₂,
or OR₂ and OR₃ can be taken together to form a six membered ring of the formula (Ia) 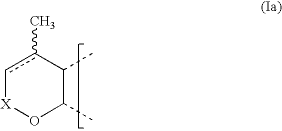
wherein X is C═O or CH(CH₃);
wherein the bracketed-dashed bonds indicate attachment to backbone;
Y is C═CH₂, C═O, CHCH₃, or CH₂;
n is 1 or 0, or
a pharmaceutically acceptable salt, ester, amide, solvate, hydrate, complex, or combination thereof.

16. A method according to claim 15 where the compound has the formula (II):

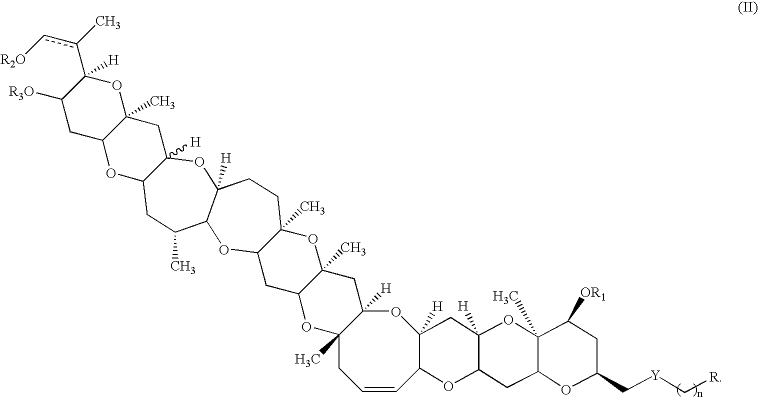

17. A method of treating ciguatera fish poisoning in a subject comprising administering to a subject in need of such treatment a therapeutically effective amount of a compound of the formula (III): 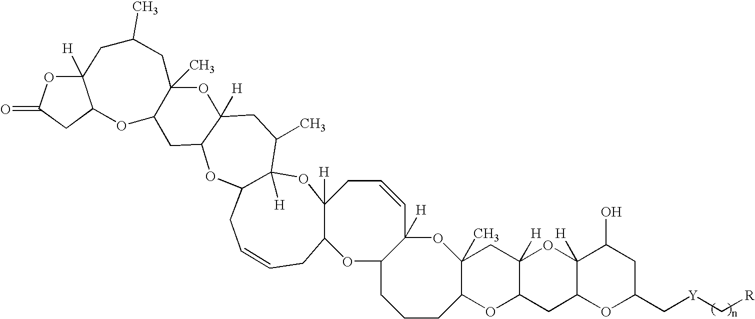
wherein
R is H, OH, halogen, C₁-C₆ lower alkyl, C₁-C₆ alkyl ester, C₂-C₆ alkenyl ester, amino, amido, aldehydo, aryl ester, cycloalkyl ester, cycloalkenyl ester, purinyl, pyrimidinyl, heterocyclyl, aryl, or heteroaryl;
Y is C═O, C═CH₂, CHCH₃ or CH₂; and
n is 1 or 0,
or a pharmaceutically acceptable salt, solvate, hydrate, ester, amide, complex, or combination thereof.

18. A method according to claim 17, where the compound has the formula (IV)

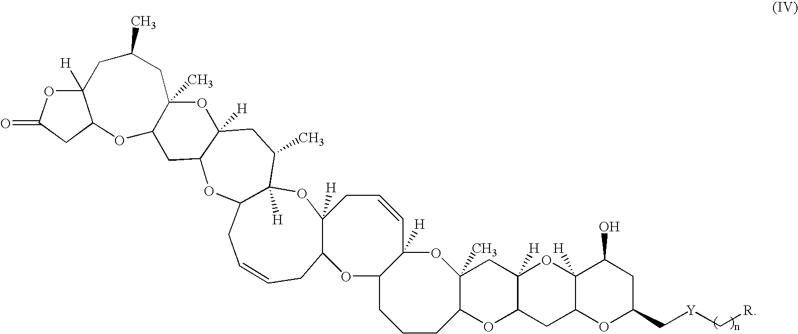

19. A method according to claim 15, wherein the therapeutically effective amount is administered in a dosage of between about 0.1 pg to about 100 mg per day.

20. A method according to claim 17, wherein the therapeutically effective amount comprises a dosage of between about 0.1 pg to about 100 mg per day.

21. A method according to claim 15, wherein the therapeutically effective amount of the compound is administered by inhalation.

22. A method according to claim 15, wherein the therapeutically effective amount of the compound is administered orally.

23. A method according to claim 15, where the compound is administered as a pharmaceutical formulation comprising the compound, or a pharmaceutically acceptable salt, solvate, amide, ester, hydrate, complex, or combination thereof, and at least one pharmaceutically acceptable carrier, excipient, solvent, diluent, or adjuvant.

24. A method according to claim 17, where the compound is administered as a pharmaceutical formulation comprising the compound, or a pharmaceutically acceptable salt, solvate, ester, amide, hydrate, complex, or combination thereof, and at least one pharmaceutically acceptable carrier, excipient, solvent, diluent, or adjuvant.

25. A method according to claim 17, wherein the therapeutically effective amount of the compound is administered by inhalation.

26. A method according to claim 17, wherein the therapeutically effective amount of the compound is administered orally.
